(19) United States
(12) Patent Application Publication
(10) Pub. No.: US 2006/0035897 A1
(43) Pub. Date: Feb. 16, 2006

(54) Trifluoromethyl substituted benzamides as kinase inhibitors

(76) Inventors: Giorgio Caravatti, Bottmingen (CH); Pascal Furet, Thann (FR); Patricia Imbach, Kaiseraugst (CH); Georg Martiny-Baron, Herbolzheim (DE); Lawrence Blas Perez, Hopkinton, MA (US); Tao Sheng, Westwood, MA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/201,348

(22) Filed: Aug. 10, 2005

(30) Foreign Application Priority Data
Aug. 11, 2004 (GB) 0417905.7

(51) Int. Cl. A61K 31/502; A61K 31/517; A61K 31/47; A61K 31/428

(52) U.S. Cl. 514/248; 514/266.1; 514/310; 514/367; 544/283; 544/237; 546/148; 548/152

(57) ABSTRACT

The invention relates to trifluoromethyl substituted benzamide compounds of the formula I, 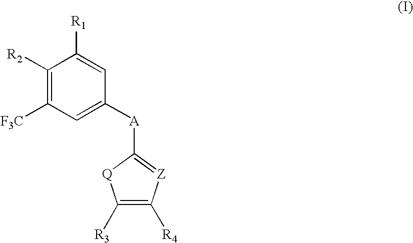 pharmaceuticals comprising these compounds, their use as or for the manufacture of pharmaceuticals, particularly as inhibitors of protein kinases, especially of ephrin receptor kinases, and/or the treatment of a condition, disorder or disease state mediated by a protein kinase activity and/or a proliferative disease, methods of treatment comprising administering the compounds, especially of therapeutic and prophylactic treatment, methods for the manufacture of the compounds and novel intermediates and partial steps for their synthesis.

### SUMMARY OF THE INVENTION

The invention relates to trifluoromethyl substituted benzamide compounds, pharmaceuticals comprising these compounds, their use as or for the manufacture of pharmaceuticals, particularly as inhibitors of protein kinases, especially of ephrin receptor kinases, such as EphB4 kinase (very preferred), c-abl, Flt-3, KDR, c-Src, c-kit, FGFR-1, c-Rat, b-Raf, cdk-1, Ins-R, Tek, KDR and/or RET kinase(s), and/or mutated forms thereof, and/or the treatment of a condition, disorder or disease state mediated by a protein kinase activity and/or a proliferative disease, methods of treatment comprising administering the compounds, especially of therapeutic and prophylactic treatment, methods for the manufacture of the compounds and novel intermediates and partial steps for their synthesis.

### BACKGROUND OF THE INVENTION

Certain fused heteroaryl derivatives have been described for use as P38 Kinase inhibitors in the treatment of e.g. rheumatoid arthritis, see WO 2004/010995. The focus of said application lies on cyclopropyl substituted derivatives.

In view of the large number of protein kinases and the multitude of proliferative and other protein kinase-related diseases, there is an ever-existing need to provide new classes of compounds that are useful as protein kinase inhibitors and thus in the treatment of these PTK (protein tyrosine kinase) related diseases. What is required are new classes of pharmaceutically advantageous PTK inhibiting compounds.

It has now surprisingly been found that compounds with (further substituted or unsubstituted) trifluoromethyl phenyl moieties instead of the cyclopropyl moieties show activity at least, preferably selectively, on one or more of the kinases mentioned below, especially those mentioned as preferred. This residue can thus be used as basis for the design of potent kinase inhibitors. In addition, they show further advantageous pharmaceutically useful properties.

### GENERAL DESCRIPTION OF THE INVENTION

It has now surprisingly been found that the novel class of trifluoromethyl substituted benzamide compounds, especially those described below, show inhibition for specific types or classes or groups of kinases, especially one or more of c-Abl, Bcr-Abl, c-Kit, c-Raf, Flt-1, Flt-3, PDGFR-kinase, c-Src, FGF-R1, FGF-R2, FGF-R3, FGF-R4, casein kinases (CK-1, CK-2, G-CK), Pak, ALK, ZAP70, Jak1, Jak2, Axl, Cdk1, cdk4, cdk5, Met, FAK, Pyk2, Syk, Insulin receptor kinase, Tie-2 or constitutively activating mutations of kinases (activating kinases) such as of Bcr-Abl, c-Kit, c-Raf, Flt-3, FGF-R3, PDGF-receptors, and/or Met. Especially preferred, they show inhibition for c-abl, c-kit, FGFR (e.g. FGFR-1), Ins-R, Tek, HER-1, more preferably c-Src, Tie/Tek, KDR kinase, c-Abl, c-Raf, b-Raf, RET-receptor kinase or Ephrin receptor kinases e.g. EphB2 kinase or related kinases, especially EphB4 kinase; or mutated forms of any one or more of these (e.g. Bcr-Abl, RET/MEN2A, RET/MEN2B, RET/PTC1-9 or b-raf(V599E)).

In view of these activities, the compounds can be used for the treatment of diseases related to especially aberrant or excessive activity of such types of kinases, especially those mentioned and most especially those mentioned as being preferred.

### DETAILED DESCRIPTION OF THE INVENTION

The invention in particular relates to trifluoromethyl substituted benzamide compounds of the formula I, 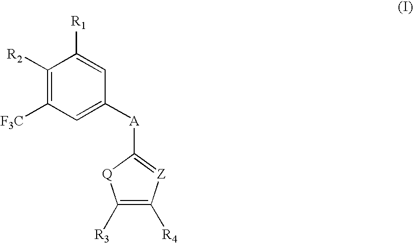
wherein
R₁ is hydrogen or —N(R₆R₇) wherein each of R₆ and R₇ is alkyl or R₆ and R₇, together with the nitrogen to which they are bound, form a 5- to 7-membered heterocyclic ring, where the additional ring atoms are selected from carbon and 0, 1 or 2 heteroatoms selected from nitrogen, oxygen and sulfur and which ring is unsubstituted or, if a further nitrogen ring atom is present, unsubstituted or substituted by alkyl at that nitrogen;
R₂ is hydrogen or —CH₂—N(R₆R₇) wherein each of R₆ and R₇ is alkyl or R₆ and R₇, together with the nitrogen to which they are bound, form a 5- to 7-membered heterocyclic ring, where the additional ring atoms are selected from carbon and 0, 1 or 2 heteroatoms selected from nitrogen, oxygen and sulfur and which ring is unsubstituted or, if a further nitrogen ring atom is present, unsubstituted or substituted by alkyl at that nitrogen;
   with the proviso, that at least one of R₁ and R₂ is hydrogen;
R₃ is halo or C₁-C₇-alkyl;
R₄ is bicyclic heterocyclyl selected from the group consisting of 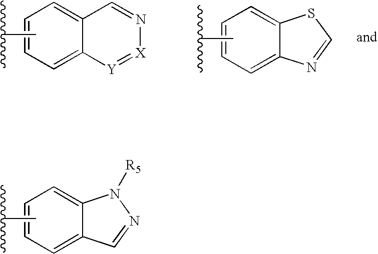
   wherein
X is CH, N or C—NH₂;
Y is CH or N;
   with the proviso that not both of X and Y are N simultaneously;
   and R₅ is hydrogen, C₁-C₇-alkyl or unsubstituted or substituted phenyl;
A is —C(═O)—NH— (with the —NH— bound to the ring comprising Q and Z in formula I) or —NH—C(═O)— (with the —C(═O)— bound to the ring comprising Q and Z in formula I);
Z is CH or N; and
Q is —S— or —CH═CH—;
   or a (preferably pharmaceutically acceptable) salt thereof where one or more salt-forming groups are present.

The present invention also relates to a method of treating a kinase dependent and/or proliferative disease comprising administering a compound of the formula I to a warm-blooded animal, especially a human, and the use of a compound of the formula I, especially for treating a kinase dependent disease or disorder. The present invention also relates to pharmaceutical preparations comprising a compound of the formula I, especially for the treatment of a kinase dependent disease or disorder, a process for the manufacture of a compound of the formula I, and novel starting materials and intermediates for their manufacture. The present invention also relates to use of a compound of formula I in the manufacture of a pharmaceutical preparation for the treatment of a kinase dependent disease.

The general terms or symbols used hereinbefore and hereinafter preferably have within, the context of this disclosure, the following meanings, unless otherwise indicated:

In each case where a waved line vertical to a bond is used, this marks the bond where a given moiety is bound to the rest of the corresponding molecule.

The term “lower” or “C₁-C₇-” defines a moiety with up to and including maximally 7, especially up to and including maximally 4, carbon atoms, said moiety being branched or straight-chained. Lower or C₁-C₇-alkyl, for example, is n-pentyl, n-hexyl or n-heptyl or preferably C₁-C₄-alkyl, especially as methyl, ethyl, n-propyl, sec-propyl, n-butyl, isobutyl, sec-butyl, tert-butyl.

Unsubstituted or substituted phenyl is unsubstituted or substituted by one or more, preferably one or two substituents, wherein the substituents are independently selected from any one or more of the functional groups including: halo, lower alkyl, substituted lower alkyl, such as halo lower alkyl e.g. trifluoromethyl, lower alkenyl, lower alkynyl, lower alkanoyl, lower alkoxy, hydroxy, etherified or esterified hydroxy, amino, mono- or disubstituted amino, such as mono- or di-lower alkylamino, amino lower alkoxy; lower alkanoylamino; amidino, nitro, cyano, cyano-lower alkyl, carboxy, esterified carboxy, especially lower alkoxy carbonyl, e.g. methoxy carbonyl, n-propoxy carbonyl or iso-propoxy carbonyl, lower alkanoyl, benzoyl, carbamoyl, N-mono- or N,N-disubstituted carbamoyl, such as N-mono- or N,N-di-lower alkylcarbamoyl or N-mono- or N,N-di-(hydroxy-lower alkyl)-carbamoyl, amidino, guanidino, ureido, mercapto, sulfo, lower alkylthio, sulfonamido, benzosulfonamido, sulfono, phenyl, phenyl-lower alkyl, such as benzyl, phenoxy, phenyl-lower alkoxy, such as benzyloxy, phenylthio, phenyl-lower alkylthio, lower alkyl-phenylthio, lower alkylsulfinyl, phenylsulfinyl, phenyl-lower alkylsulfinyl, alkylphenylsulfinyl, lower alkanesulfonyl, phenylsulfonyl, phenyl-lower alkylsulfonyl, alkylphenylsulfonyl, halogen-lower alkylmercapto, halogen-lower alkylsulfonyl, such as trifluoromethane sulfonyl, dihydroxybora (—B(OH)₂), lower alkylene dioxy bound at adjacent C-atoms of the ring, such as methylene dioxy, phosphono (—P(═O)(OH)₂), hydroxy-lower alkoxy phosphoryl or di-lower alkoxyphosphoryl, or —NRₐR_{b}, wherein Rₐ and R_{b} can be the same or different and are independently H; lower alkyl (e.g. methyl, ethyl or propyl); or Rₐ and R_{b} together with the N atom form a 3- to 8-membered heterocyclic ring containing 1-4 nitrogen, oxygen or sulfur atoms (e.g. piperazinyl, lower alkyl-piperazinyl, azetidinyl, pyrrolidinyl, piperidino, morpholinyl, imidazolinyl).

Alkyl preferably has 1 to 12 carbon atoms or is especially lower alkyl with up to 7 carbon atoms, preferably from 1 to and including 5, and is linear or branched; preferably, lower alkyl as defined above.

Halo or halogen is preferably fluoro, chloro, bromo or iodo, most preferably fluoro, chloro or bromo.

Salts are especially the pharmaceutically acceptable salts of compounds of formula I. They can be formed where salt forming groups, such as basic or acidic groups, are present that can exist in dissociated form at least partially, e.g. in a pH range from 4 to 10 in aqueous solutions, or can be isolated especially in solid form.

Such salts are formed, for example, as acid addition salts, preferably with organic or inorganic acids, from compounds of formula I with a basic nitrogen atom, especially the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, lactic acid, fumaric acid, succinic acid, citric acid, amino acids, such as glutamic acid or aspartic acid, maleic acid, hydroxymaleic acid, methylmaleic acid, benzoic acid, methane- or ethane-sulfonic acid, ethane-1,2-disulfonic acid, benzenesulfonic acid, 2-naphthalenesulfonic acid, 1,5-naphthalene-disulfonic acid, N-cyclohexylsulfamic acid, N-methyl-, N-ethyl- or N-propyl-sulfamic acid, or other organic protonic acids, such as ascorbic acid.

In the presence of negatively charged radicals, such as carboxy or sulfo, salts may also be formed with bases, e.g. metal or ammonium salts, such as alkali metal or alkaline earth metal salts, for example sodium, potassium, magnesium or calcium salts, or ammonium salts with ammonia or suitable organic amines, such as tertiary monoamines, for example triethylamine or tri(2-hydroxyethyl)amine, or heterocyclic bases, for example N-ethyl-piperidine or N,N′-dimethylpiperazine.

When a basic group and an acid group are present in the same molecule, a compound of formula I may also form internal salts.

For isolation or purification purposes it is also possible to use pharmaceutically unacceptable salts, for example picrates or perchlorates. For therapeutic use, only pharmaceutically acceptable salts or free compounds are employed (where applicable comprised in pharmaceutical preparations), and these are therefore preferred.

In view of the close relationship between the compounds in free form and in the form of their salts, including those salts that can be used as intermediates, for example in the purification or identification of the compounds or salts thereof, any reference to “compounds” hereinbefore and hereinafter, especially to the compound(s) of the formula I, is to be understood as referring also to one or more salts thereof or a mixture of a free compound and one or more salts thereof, as appropriate and expedient and if not mentioned otherwise.

Where the plural form is used for compounds, salts, pharmaceutical preparations, diseases, disorders and the like, this is intended to mean also a single compound, salt, pharmaceutical preparation, disease or the like, and vice versa.

The compounds of formula I have valuable pharmacological properties and are useful in the treatment of kinase dependent diseases, e.g., as drugs to treat one or more proliferative diseases.

The terms “treatment” or “therapy” (especially of tyrosine protein kinase dependent diseases or disorders) refer to the prophylactic or preferably therapeutic (including but not limited to palliative, curing, symptom-alleviating, symptom-reducing, kinase-regulating and/or kinase-inhibiting) treatment of said diseases, especially of the diseases mentioned below.

Where subsequently or above the term “use” is mentioned (as verb or noun) (relating to the use of a compound of the formula I or a pharmaceutically acceptable salt thereof), this (if not indicated differently in the context) includes any one or more of the following embodiments of the invention, respectively (if not stated otherwise): the use in the treatment of a (especially tyrosine) protein kinase dependent disease, the use for the manufacture of pharmaceutical compositions for use in the treatment of a protein kinase dependent disease, methods of use of one or more compounds of the formula I in the treatment of a protein kinase dependent and/or proliferative disease, pharmaceutical preparations comprising one or more compounds of the formula I for the treatment of a protein kinase dependent disease, and one or more compounds of the formula I in the treatment of a protein kinase dependent disease, as appropriate and expedient, if not stated otherwise. In particular, diseases to be treated and are thus preferred for “use” of a compound of formula I are selected from (especially tyrosine) protein kinase dependent (“dependent” meaning also “supported”, not only “solely dependent”) diseases mentioned below, especially proliferative diseases mentioned below, more especially any one or more of these or other diseases that depend on one or more of c-Abl, Bcr-Abl, c-Kit, c-Raf, Flt-1, Flt-3, PDGFR-kinase, c-Src, FGF-R1, FGF-R2, FGF-R3, FGF-R4, casein kinases (CK-1, CK-2, G-CK), Pak, ALK, ZAP70, Jak1, Jak2, Axl, Cdk1, cdk4, cdk5, Met, FAK, Pyk2, Syk, Insulin receptor kinase, Tie-2 or constitutively activating mutations of kinases (activating kinases) such as of Bcr-Abl, c-Kit, c-Raf, b-Raf, Flt-3, FGF-R3, PDGF-receptors and/or Met, (hereinafter “said kinases”) and more especially depend on c-Raf, b-Raf, c-src, c-Abl, Tie/Tek and most especially on KDR, RET-receptor kinase, and/or preferably Ephrin receptor kinase (e.g., EphB2 kinase or related Eph kinases, most preferably EphB4 kinase), or a mutant of any one or more of these, and a compound of the formula I can therefore be used in the treatment of a kinase dependent disease, especially one or more diseases depending on the kinases mentioned above and below, where (especially in the case of aberrantly highly-expressed, constitutively activated and/or mutated kinases) said kinase-dependent disease or disease is dependent on the activity of the said kinase pathways or any combination of two or more of the mentioned kinases.

Where a kinase dependent disease or disorder is mentioned, this refers preferably to any one or more of c-Abl, c-kit, FGFR (e.g. FGFR-1), c-Raf, b-Raf, c-Src, Tie/Tek, c-abl and most especially KDR, RET-receptor kinase, and/or preferably Ephrin receptor kinase (e.g., EphB2 kinase or related Eph kinases, most preferably EphB4 kinase) receptor kinase dependent diseases or disorders or diseases or disorders depending on any one or more mutant forms of these kinases, in a broader sense to the kinases mentioned above and/or below.

The compounds of formula I have valuable pharmacological properties and can be used in the treatment of protein kinase dependent diseases, e.g., as drugs to treat proliferative diseases.

In the following description of typical exemplary testing systems, the following abbreviations have the following meanings: DMSO=dimethyl sulfoxide; DTT=dithiothreitol; EDTA=ethylene diamine tetraacetate; MOI=multipliciy of infection; PMSF=p-toluenesulfonyl fluoride; Tris=tris(hydroxymethyl)aminomethane. An “inhibitor” is a test compound of the formula I if not mentioned otherwise.

The (especially important and preferred) efficacy of compounds of the formula I as inhibitors or Ephrin B4 receptor (EphB4) kinases can be demonstrated as follows: EphB4: Production and measure of activity:

Generation of Bac-to-Bac™ (Initrogen Life Technologies, Basel, Switzerland) GST-fusion expression vectors: Entire cytoplasmatic coding regions of the EphB-class are amplified by PCR from cDNA libraries derived from human placenta or brain, respectively. Recombinant baculovirus are generated that express the amino acid region 566-987 of the human EphB4 receptor (SwissProt Database, Accession No. P54760). GST sequence is cloned into pFastBacl® vector (Invitrogen Life Technologies, Basel, Switzerland) and PCR amplified. cDNAs encoding EphB4—receptor domains, respectively, are cloned in frame 3′prime to the GST sequence into this modified FastBac1 vector to generate pBac-to-Bac™ donor vectors. Single colonies arising from the transformation are inoculated to give overnight cultures for small scale plasmid preparation. Restriction enzyme analysis of plasmid DNA reveals several clones to contain inserts of the expected size. By automated sequencing the inserts and approximately 50 bp of the flanking vector sequences are confirmed on both strands. Production of viruses: Viruses for each of the kinases are made according to the protocol supplied by GIBCO if not stated otherwise. In brief, transfer vectors containing the kinase domains are transfected into the DH10Bac cell line (GIBCO) and plated on selective agar plates. Colonies without insertion of the fusion sequence into the viral genome (carried by the bacteria) are blue. Single white colonies are picked and viral DNA (bacmid) isolated from the bacteria by standard plasmid purification procedures. Sf9 cells or Sf21 cells are then transfected in 25 cm² flasks with the viral DNA using Cellfectin reagent according to the protocol. Purification of GST-tagged kinases: The centrifuged cell lysate is loaded onto a 2 mL glutathione-sepharose column (Pharmacia) and washed three times with 10 mL of 25 mM Tris-HCl, pH 7.5, 2 mM EDTA, 1 mM DTT, 200 mM NaCl. The GST-tagged proteins are then eluted by 10 applications (1 mL each) of 25 mM Tris-HCl, pH 7.5, 10 mM reduced-glutathione, 100 mM NaCl, 1 mM DTT, 10% Glycerol and stored at −70° C.

Protein kinase assays: The activities of protein kinases are assayed in the presence or absence of inhibitors, by measuring the incorporation of ³³P from [γ³³P]ATP into a polymer of glutamic acid and tyrosine (poly(Glu,Tyr)) as a substrate. The kinase assays with purified GST-EphB (30 ng) are carried out for 15-30 min at ambient temperature in a final volume of 30 μL containing 20 mM Tris.HCl, pH 7.5, 10 mM MgCl₂, 3-50 mM MnCl₂, 0.01 mM Na₃VO₄, 1% DMSO, 1 mM DTT, 3 μg/mL poly(Glu,Tyr) 4:1 (Sigma; St. Louis, Mo., USA) and 2.0-3.0 μM ATP (γ-[³³P]-ATP 0.1 μCi). The assay is terminated by the addition of 20 μL of 125 mM EDTA. Subsequently, 40 μl of the reaction mixture are transferred onto Immobilon-PVDF membrane (Millipore, Bedford, Mass., USA) previously soaked for 5 min with methanol, rinsed with water, then soaked for 5 min with 0.5% H₃PO₄ and mounted on vacuum manifold with disconnected vacuum source. After spotting all samples, vacuum is connected and each well rinsed with 200 μl 0.5% H₃PO₄. Membranes are removed and washed 4× on a shaker with 1.0% H₃PO₄, once with ethanol. Membranes are counted after drying at ambient temperature, mounting in Packard TopCount96-well frame, and addition of 10 μL/well of Microscint™ (Packard). IC₅₀ values are calculated by linear regression analysis of the percentage inhibition of each compound in duplicate, at four concentrations (usually 0.01, 0.1, 1 and 10 μM). One unit of protein kinase activity is defined as 1 nmole of ³³P ATP transferred from [γ³³P] ATP to the substrate protein per minute per mg of protein at 37° C. Compounds of formula I show EphB4 inhibition down to 10 nM, preferably IC₅₀ values between 0.01-1.0 μM.

The efficacy of the compounds of the invention as inhibitors of KDR protein-tyrosine kinase activity can be demonstrated as follows: The inhibition of VEGF-induced receptor autophosphorylation can be confirmed in cells such as transfected CHO cells, which permanently express human VEGF-R2 receptor (KDR), and are seeded in complete culture medium (with 10% fetal calf serum=FCS) in 6-well cell-culture plates and incubated at 37° C. under 5% CO₂ until they show about 80% confluency. The compounds to be tested are then diluted in culture medium (without FCS, with 0.1% bovine serum albumin) and added to the cells. Controls comprise medium without test compounds. After 2 h incubation at 37° C., recombinant VEGF is added; the final VEGF concentration is 20 ng/ml. After a further incubation period of five minutes at 37° C., the cells are washed twice with ice-cold PBS (phosphate-buffered saline) and immediately lysed in 100 μl, lysis buffer per well. The lysates are then centrifuged to remove the cell nuclei, and the protein concentrations of the supernatants are determined using a commercial protein assay (BIORAD). The lysates can then either be immediately used or, if necessary, stored at −20° C. Using this protocol, the compounds of the formula I can be found to show IC₅₀ values for KDR inhibition in the range from 0.005-20 μM, preferably in the range from 0.005 to 20 μM, more preferably in the range from 0.005 to 0.5 μM. The inhibition of RET can be measured as follows: The baculovirus donor vector pFB-GSTX3 is used to generate a recombinant baculovirus that expresses the amino acid region 658-1072 (Swiss prot No. Q9BTB0) of the intra-cytoplasmic kinase domain of human RET-Men2A which corresponds to the wild-type kinase domain of RET (wtRET) and RET-Men2B, which differs from the wtRET by the activating mutation in the activation loop M918T (D. S. Acton et al., Oncogene 19, 3121 (2000)). The coding sequences for the cytoplasmic domain of wtRET and RET-Men2B are amplified by PCR from the plasmids pBABEpuro RET-Men2A and pBABEpuro RET-Men2B. The amplified DNA fragments and the pFB-GSTX3 vector are made compatible for ligation by digestion with SalI and KpnI. Ligation of these DNA fragments results in the baculovirus donor plasmid pFB-GX3-RET-Men2A and pFB-GX3-RET-Men2B, respectively.

Production of virus: Transfer vectors containing the kinase domains are transfected into the DH10Bac cell line (GIBCO) and plated on selective agar plates. Colonies without insertion of the fusion sequence into the viral genome (carried by the bacteria) are blue. Single, white colonies are picked and viral DNA (bacmid) are isolated from the bacteria by standard plasmid purification procedures. Sf9 cells or Sf21 (American Type Culture Collection) cells are then transfected in 25 cm² flasks with the viral DNA using Cellfectin reagent. Determination of small scale protein expression in Sf9 cells: Virus-containing medium is collected from the transfected cell culture and used for infection to increase its titer. Virus-containing media obtained after two rounds of infection are used for large-scale protein expression. For large-scale protein expression, 100 cm² round tissue culture plates are seeded with 5×10⁷ cells/plate and infected with 1 mL of virus-containing medium (approximately 5 MOIs). After 3 days, the cells are scraped off the plate and centrifuged at 500 rpm for 5 minutes. Cell pellets from 10-20 of the 100 cm² plates, are re-suspended in 50 mL of ice-cold lysis buffer (25 mM tris-HCl, pH 7.5, 2 mM EDTA, 1% NP-40, 1 mM DTT, 1 mM PMSF). The cells are stirred on ice for 15 min and then centrifuged at 5,000 rpms for 20 minutes.

Purification of GST-tagged proteins: The centrifuged cell lysate is loaded onto a 2 mL glutathione-sepharose column (Pharmacia) and is washed 3× with 10 mL of 25 mM tris-HCl, pH 7.5, 2 mM EDTA, 1 mM DTT, 200 mM NaCl. The GST-tagged proteins are then eluted by 10 applications (1 mL each) of 25 mM tris-HCl, pH 7.5, 10 mM reduced-glutathione, 100 mM NaCl, 1 mM DTT, 10% glycerol and stored at −70° C.

Measurement of enzyme activity: Tyrosine protein kinase assays with either purified GST-wtRET or GST-RET-Men2B protein are carried out in a final volume of 30 μL containing 15 ng of either GST-wtRET or GST-RET-Men2B protein, 20 mM tris-HCl, pH 7.5, 1 mM MnCl₂, 10 mM MgCl₂, 1 mM DTT, 3 μg/mL poly(Glu,Tyr) 4:1, 1% DMSO, 2.0 μM ATP (γ-[³³P]-ATP 0.1 μCi). The activity is assayed in the presence or absence of inhibitors, by measuring the incorporation of ³³P from [γ³³P] ATP into poly(Glu,Tyr) 4:1. The assay is carried out in 96-well plates at ambient temperature for 15 minutes under conditions described below and terminated by the addition of 20 μL of 125 mM EDTA. Subsequently, 40 μL of the reaction mixture are transferred onto Immobilon-PVDF membrane (Millipore) previously soaked for 5 minutes with methanol, rinsed with water, then soaked for 5 minutes with 0.5% H₃PO₄ and mounted on vacuum manifold with disconnected vacuum source. After spotting all samples, vacuum is connected and each well-rinsed with 200 μL 0.5% H₃PO₄. Membranes are removed and washed 4× on a shaker with 1.0% H₃PO₄, once with ethanol. Membranes are counted after drying at ambient temperature, mounting in Packard TopCount 96-well frame, and addition of 10 μL/well of Microscint™ (Packard). IC₅₀ values are calculated by linear regression analysis of the percentage inhibition of each compound in duplicate, at 4 concentrations (usually 0.01, 0.1, 1 and 10 μM). One unit of protein kinase activity is defined as 1 nmole of ³³P ATP transferred from [γ³³P] ATP to the substrate protein/minute/mg of protein at 37° C.

IC₅₀ Calculations:

Input: 3×4 μL stopped assay on Immobilon membrane, not washed.
Background (3 wells): assay with H₂O instead of enzyme.
Positive control (4 wells): 3% DMSO instead of compound.
Bath control (1 well): no reaction mix.

IC₅₀ values are calculated by logarithmic regression analysis of the percentage inhibition of each compound at 4 concentrations (usually 3- or 10-fold dilution series starting at 10 μM). In each experiment, the actual inhibition by reference compound is employed for normalization of IC₅₀ values to the basis of an average value of the reference inhibitor:
Normalized IC₅₀=measured IC₅₀ average ref. IC₅₀/measured ref. IC₅₀
Example
   Reference inhibitor in experiment 0.4 μM, average 0.3 μM;
   Test compound in experiment 1.0 μM, normalization: 0.3/0.4=0.75 μM.

For example, staurosporine or a synthetic staurosporine derivative are used as reference compounds. Using this protocol, the compounds of the formula I can be found to show IC₅₀ values for RET inhibition in the range from 0.001-10 μM, preferably in the range from 0.01-1 μM.

The compounds of formula I also inhibit other tyrosine protein kinases such as especially the c-Src kinase, c-Kit and/or FGFR; all of which play a part in growth regulation and transformation in animal, especially mammal cells, including human cells. An appropriate assay is described in Andrejauskas-Buchdunger et al., Cancer Res. 52, 5353-8 (1992). Using this test system, compounds of the formula I can show IC₅₀ values for inhibition of c-Src in the range of 0.005 to 100 μM, usually between 0.01 and 5 μM. Compounds of formula I also can show IC₅₀ values for c-kit inhibition in the range of 0.01 to 5 μM, usually between 0.005 and 5 μM.

The inhibition of Tek can be determined as follows: The procedure of the expression, purification and assay these kinases has been described. Fabbro et al., Pharmacol. Ther. 82(2-3) 293-301 (1999). In brief, the glutathione S-transferase (GST) gene from the pAcG1 vector (Pharmingen) is excised with EcoRV and EcoRI and inserted into the cloning site of the Fast-Bac baculoviral vector (GIBCO) creating a 5530 bp vector with N-terminal cloning sites derived from the pAcG1 fusion vector (FBG0). The C-terminal cloning site may be any cloning site (from the Fast-Bac vector) downstream of the N-terminal cloning site used. N-terminally GST-fused (pAcG1, Pharmingen) KDR or Tek kinase domains are obtained from ProQinase, Freiburg, Germany. Tek is recloned into the FBG1 vector by EcoRI excision and ligation into EcoRI digested FBG1 (FBG1-Tek). The coding sequences for the whole cytoplasmic domain of c-Kit (aa 544-976) and c-Fms (aa 538-972) are amplified by PCR from human uterus and from human bone marrow cDNA libraries (Clontech), respectively. The amplified DNA fragments are fused to GST by cloning them into FBG1 as BamHI-EcoRI insertions, to yield FBG1-c-Kit and FBG1-c-Fms. Tek is recloned into the FBGO transfer vector by EcoRI excision and ligation into EcoRI digested FBGO (FBG-Tie2/Tek). FGFR-1 and c-met kinase domains are obtained by PCR from human A431 cells. N-terminal primers contain an overhanging EcoRI site, while C-terminal primers contain a XhoI site to aid cloning into the transfer vectors. After digestion of both the PCR fragments and FBGO the cleavage products are gel-purified and ligated together to form the kinase constructs (FBG-Met, FBG-FGFR-1).

Viruses for the kinases are made according to the protocol supplied by GIBCO. In brief, transfer vectors containing the kinase domains are transfected into the DH10Bac cell line (GIBCO), plated on agar plates containing the recommended concentrations of Blue-Gal, IPTG, Kanamycin, Tetracycline, and Gentamycin. Colonies without insertion of the fusion sequence into the viral genome (carried by the bacteria) are blue. A single white colony is usually picked and viral DNA (bacmid) isolated from the bacteria by standard plasmid mini prep procedures. Sf9 cells or High Five cells (GIBCO) are then transfected in 25 cm² flasks with the viral DNA using the Cellfectin reagent and protocol supplied with the Bac-to-Bac kit (GIBCO). Virus containing media is collected from the transfected cell culture and used for infection to increase its titer. Virus containing media obtained after two rounds of infection is used for large-scale protein expression. For large-scale protein expression 100 cm² round tissue culture plates are seeded with 5×10⁷ cells/plate and infected with 1 ml of virus-containing media (about 5 MOIs). After 3 days the cells are scraped off the plate and centrifuged at 500 rpm for 5 min.

Cell pellets from 10-20, 100 cm² plates, are resuspended in 50 ml of ice-cold lysis buffer (25 mM Tris-HCl, pH 7.5, 2 mM EDTA, 1% NP-40, 1 mM DTT, 1 mM PMSF). The cells are stirred on ice for 15 min and then centrifuged at 5000 rpms for 20 min. The supernatant is loaded onto a 2 ml glutathione-sepharose column and washed three times with 10 ml of 25 mM Tris-HCl, pH 7.5, 2 mM EDTA, 1 mM DTT, 200 mM NaCl. The GST-tagged proteins are then eluted by 10 applications (1 ml each) of 25 mM Tris-HCl, pH 7.5, 10 mM reduced-glutathione, 100 mM NaCl, 1 mM DTT, 10% glycerol and stored at −70° C.

The assays (30 μl) contain 200-1800 ng of enzyme protein (depending on the specific activity), 20 mM Tris-HCl, pH 7.6, 3 mM MnCl₂, 3 mM MgCl₂ 1 mM DTT, 10 μM Na₃VO₄, 3 μg/ml poly(Glu,Tyr) 4:1, 8 μM ATP (γ-[³³P]-ATP 0.1 μCi). Reactions are incubated for 20 min at ambient temperature and then stopped by addition of 25 μl 0.25 M EDTA (pH 7.0). An aliquot of 40 μl is spotted with a multichannel dispenser on Immobilon P membranes mounted in a Millipore Microtiter filter manifold connected to a low vacuum source. After elimination of liquid, the membrane is transferred to a sequence of 4 washing baths containing 0.5% H₃PO₄ and one with EtOH (shaking incubation for 10 min each), dried, mounted onto a Hewlett Packard TopCount manifold added 10 μl Microscint® and counted. Compounds of formula I can show IC₅₀ values, calculated by linear regression analysis, for Tek inhibition of about 0.01-100 μM, preferably 0.1 to 10 μM.

The inhibition of c-Raf-1 can be determined as follows: Production of recombinant c-Raf-1 protein, is obtained by triple infection of Sf21 cells with GST-c-Raf-1 recombinant baculovirus together with v-Src and v-Ras recombinant baculoviruses that are required for active c-Raf-1 kinase production (Williams et al., PNAS 1992; 89: 2922-2926). Active Ras (v-Ras) is required to recruit c-Raf-1 to the cell membrane and v-Src to phosphorylate c-Raf-1 to fully activate it (Williams et al., PNAS 1992; 89: 2922-2926). Cells are seeded at 2.5×10⁷ cells per 150 mm dish and allowed to attach to a 150 mm dish for 1 hr at room temperature (RT). Media (SF90011 containing 10% FBS) is aspirated and recombinant baculovirus; GST-C-Raf-1, v-Ras and v-Src are added at MOI of 3.0, 2.5 and 2.5, respectively, in a total volume of 4-5 mL. Cells are incubated for 1 hr at RT and then 15 mL of medium is added. Infected cells are incubated for 48-72 hr at 27° C. Infected Sf21 cells are scraped and collected into a 50 mL tube and centrifuged for 10 min at 4° C. at 1100 g in a Sorvall centrifuge. The cell pellet is washed once with ice cold PBS and lysed with 0.6 mL lysis buffer per 2.5×10⁷ cells. Complete lysis of cells is achieved after 10 min on ice with occasional pipetting. The cell lysates are centrifuged for 10 min at 4° C. at 14,500 g in a Sorvall centrifuge with SS-34 rotor and the supernatant is transferred to a fresh tube and stored at −80° C. c-Raf-1 is purified from cell lysates using 100 μL of packed Glutathione-Sepharose 4B beads equilibrated in ice cold PBS per 2.5×10⁷ cells. GST-c-Raf-1 is allowed to bind to the beads at 4° C. for 1 hr with rocking. Bound GST-c-Raf-1 with beads is transferred to a column. The column is washed once with lysis buffer and twice with ice cold Tris buffered saline. Ice cold elution buffer is added and column flow is stopped to allow the free glutathione to disrupt the interaction of GST-c-Raf-1 with glutathione sepharose beads. Fractions (1 mL) are collected into pre-chilled tubes. Each tube contains 10% glycerol (final concentration) to maintain kinase activity during freeze thaw cycles. Purified fractions of GST-c-Raf-1 kinase protein are stored at −80° C.

IκB was used as substrate for the c-Raf-1 kinase. IκB is expressed in bacteria as a His-tagged protein (cloned and kindly provided by Dr. Eder; ABM, Novartis, Basel). BL21 LysS bacteria containing the IκB plasmid are grown to an OD₆₀₀ of 0.6 in LB medium then induced to express the kb with IPTG (final concentration of 1 mM) for 3 hrs at 37° C. and then bacteria are lysed by sonication (microtip limit setting for 3 times at 1 min each in sonication buffer [50 mM Tris pH 8.0, 1 mM DTT, 1 mM EDTA] and centrifuged at 10,000 g for 15 min. The supernatant is mixed with ammonium sulfate to give a final concentration of 30%. This mixture is rocked for 15 min at 4° C. then spun at 10,000 g for 15 min. The pellet is resuspended in binding buffer (Novagen) containing 10 mM BSA. This solution is applied to Ni-agarose (Novagen) and washed according to the Novagen manual. IκB is eluted from the column using elution buffer (0.4 M imidazole, 0.2 M NaCl, 8 mM Tris pH 7.9). Fractions containing protein are dialyzed in 50 mM Tris pH 8,1 mM DTT.

The activity of c-Raf-1, b-Raf and of b-Raf(V599E) protein kinases is assayed in the presence or absence of inhibitors, by measuring the incorporation of ³³P from [γ³³P] ATP into IκB. The assay is carried out in 96-well plates at ambient temperature for 60 min. It contains (total volume of 30 μl): c-raf-1, b-Raf or b-Raf(V599E) kinase (400-600 ng), 25 mM Tris-HCl, pH 7.5, 5 mM MgCl₂, 5 mM MnCl₂, 10 μM Na₃VO₄, 1 mM OTT and 0.3 μCi/assay [γ³³ P]-ATP (10 μM ATP) using 600 ng IκB in the presence of 1% DMSO. Reactions are terminated by adding 10 μL of 250 mM EDTA and 30 μL of the reaction mixture is transferred onto Immobilon-PVDF membrane (Millipore, Bedford, Mass., USA) previously soaked for 5 min with methanol, rinsed with water, then soaked for 5 min with 0.5% H₃PO₄ and mounted on vacuum manifold with disconnected vacuum source. After spotting all samples, vacuum is connected and each well rinsed with 200 μL 0.5% H₃PO₄. Membranes are removed and washed 4× on a shaker with 0.5% H₃PO₄, once with ethanol. Membranes are counted after drying at ambient temperature, mounting in Packard TopCount 96-well frame, and addition of 10 μL/well of Microscint™ (Packard). Compounds of formula I can show c-Raf-1, b-Raf or b-Raf(V599E) inhibition in the range between 0.01-50 μM, preferably between 0.01 and 10 μM.

The efficacy of the compounds of the invention as inhibitors of c-Abl protein-tyrosine kinase activity can be demonstrated as follows:

An in vitro enzyme assay is performed in 96-well plates as a filter binding assay as described by Geissler et al. in Cancer Res. 1992; 52:4492-4498, with the following modifications. The His-tagged kinase domain of c-Abl is cloned and expressed in the baculovirus/Sf9 system as described by Bhat et al. in J. Biol. Chem. 1997; 272:16170-16175. A protein of 37 kD (c-Abl kinase) is purified by a two-step procedure over a Cobalt metal chelate column followed by an anion exchange column with a yield of 1-2 mg/L of Sf9 cells (Bhat et al., reference cited). The purity of the c-Abl kinase is >90% as judged by SDS-PAGE after Coomassie blue staining. The assay contains (total volume of 30 μL): c-Abl kinase (50 ng), 20 mM Tris-HCl, pH 7.5, 10 mM MgCl₂, 10 μM Na₃VO₄, 1 mM DTT and 0.06 μCi/assay [γ³³ P]-ATP (5 μM ATP) using 30 μg/mL poly-Ala,Glu,Lys,Tyr-6:2:5:1 (Poly-AEKY, Sigma P1152) in the presence of 1% DMSO. Reactions are terminated by adding 10 μL of 250 mM EDTA and 30 μL of the reaction mixture is transferred onto Immobilon-PVDF membrane (Millipore, Bedford, Mass., USA) previously soaked for 5 min with methanol, rinsed with water, then soaked for 5 min with 0.5% H₃PO₄ and mounted on vacuum manifold with disconnected vacuum source. After spotting all samples, vacuum is connected and each well rinsed with 200 μL 0.5% H₃PO₄. Membranes are removed and washed on a shaker with 0.5% H₃PO₄ (4 times) and once with ethanol. Membranes are counted after drying at ambient temperature, mounting in Packard TopCount 96-well frame, and addition of 10 μL/well of Microscint™ (Packard). Using this test system, compounds of the formula I can show IC₅₀ values of inhibition for c-Abl inhibition in the range of 0.002 to 100 μM, usually between 0.002 and 5 μM.

There are also experiments to demonstrate the antitumor activity of compounds of the formula I in vivo.

For example, in order to test whether a compound of the formula I, e.g. that of Example 1 given below, inhibits VEGF-mediated angiogenesis in vivo, its effect on the angiogenic response induced by VEGF in a growth factor implant model in mice is tested: A porous Teflon chamber (volume 0.5 mL) is filled with 0.8% w/v agar containing heparin (20 units/ml) with or without growth factor (2 μg/ml human VEGF) is implanted subcutaneously on the dorsal flank of C57/C6 mice. The mice are treated with the test compound (e.g. 25, 50 or 100 mg/kg p.o. once daily) or vehicle starting on the day of implantation of the chamber and continuing for 4 days after. At the end of the treatment, the mice are killed, and the chambers are removed. The vascularized tissue growing around the chamber is carefully removed and weighed, and the blood content is assessed by measuring the hemoglobin content of the tissue (Drabkins method; Sigma, Deisenhofen, Germany). It has been shown previously that these growth factors induce dose-dependent increases in weight and blood content of this tissue growing (characterized histologically to contain fibroblasts and small blood vessels) around the chambers and that this response is blocked by antibodies that specifically neutralize VEGF (see Wood J M et al., Cancer Res. 60(8), 2178-2189, (2000); and Schlaeppi et al., J. Cacner Res. Clin. Oncol. 125, 336-342, (1999)). With this model, inhibition can be shown in the case of compounds of the formula I.

In a broader sense of the invention, a kinase dependant disease where a compound of the formula I can be used may be a proliferative disease including a hyperproliferative condition, such as leukemias, hyperplasias, fibrosis (especially pulmonary, but also other types of fibrosis, such as renal fibrosis), angiogenesis, psoriasis, atherosclerosis and smooth muscle proliferation in the blood vessels, such as stenosis or restenosis following angioplasty. Further, a compound of the formula I may be used for the treatment of thrombosis, psoriasis, scleroderma and fibrosis.

Preferably, a compound of the formula I can be used in the therapy (including prophylaxis) of a proliferative disorder selected from tumor or cancer diseases, especially against preferably a benign or especially malignant tumor or cancer disease, more preferably carcinoma of the brain, kidney, liver, adrenal gland, bladder, breast, stomach (especially gastric tumors), ovaries, colon, rectum, prostate, pancreas, lung, vagina, thyroid, sarcoma, glioblastomas, multiple myeloma or gastrointestinal cancer, especially colon carcinoma or colorectal adenoma, or a tumor of the neck and head, an epidermal hyperproliferation, especially psoriasis, prostate hyperplasia, a neoplasia, especially of epithelial character, preferably mammary carcinoma, or a leukemia.

Compounds of formula I can be used to bring about the regression of tumors and to prevent the formation of tumor metastases and the growth of (also micro)metastases. In addition they can be used in epidermal hyperproliferation (e.g. psoriasis), in prostate hyperplasia, and in the treatment of neoplasias, especially of epithelial character, for example mammary carcinoma. It is also possible to use the compounds of formula I in the treatment of diseases of the immune system insofar as several or, especially, individual tyrosine protein kinases are involved; furthermore, the compounds of formula I can be used also in the treatment of diseases of the central or peripheral nervous system where signal transmission by at least one tyrosine protein kinase, especially selected from those mentioned specifically, is involved.

In chronic myelogeous leukemia (CML), a reciprocally balanced chromosomal translocation in hematopoietic stem cells (HSCs) produces the BCR-ABL hybrid gene. The latter encodes the oncogenic Bcr-Abl fusion protein. Whereas ABL encodes a tightly regulated protein tyrosine kinase, which plays a fundamental role in regulating cell proliferation, adherence and apoptosis, the BCR-ABL fusion gene encodes as constitutively activated kinase which transforms HSCs to produce a phenotype exhibiting deregulated clonal proliferation, reduced capacity to adhere to the bone marrow stroma and a reduced apoptotic response to mutagenic stimuli, which enable it to accumulate progressively more malignant transformations. The resulting granulocytes fail to develop into mature lymphocytes and are released into the circulation, leading to a deficiency in the mature cells and increased infection susceptibility. ATP-competitive inhibitors of Bcr-Abl have been described that prevent the kinase from activating mitogenic and anti-apoptotic pathways (e.g. P-3 kinase and STAT5), leading to the death of the BCR-ABL phenotype cells and thus providing an effective therapy against CML. The pyrazolo[1,5a]pyrimidin-7-yl amine derivatives useful according to the present invention, especially the compounds of formula I, as Bcr-Abl inhibitors are thus especially appropriate for the therapy of diseases related to its overexpression, especially leukemias, such as leukemias, e.g. CML or ALL.

The compounds of formula I are capable of slowing down tumor growth or effecting tumor regression and of preventing the formation of tumor metastases and the growth of micrometastases. They can be used especially in the case of epidermal hyperproliferation (psoriasis), in the treatment of solid cancers like for example (e.g. non-small cell) lung cancer, squameous carcinoma (head and neck), breast, gastric, ovarian, colon and prostate cancers as well as gliomas and in the treatment of leukemias, such as especially acute myeloid leukemia (AML) and chronic myeloid leukemia (CML). In addition, the compounds of formula I can be used in the treatment of disorders of the immune system in which several or, especially, individual protein tyrosine kinases and/or (furthermore) serine/threonine protein kinases are involved; the compounds of formula I can also be used in the treatment of those disorders of the central or peripheral nervous system in which signal transmission by several or, especially, a single protein tyrosine kinase(s) and/or (furthermore) serine/threonine protein kinase(s) is/are involved.

Angiogenesis is regarded as an absolute prerequisite for those tumors which grow beyond a maximum diameter of about 1-2 mm; up to this limit, oxygen and nutrients may be supplied to the tumor cells by diffusion. Every tumor, regardless of its origin and its cause, is thus dependent on angiogenesis for its growth after it has reached a certain size. Three principal mechanisms play an important role in the activity of angiogenesis inhibitors against tumors: 1) Inhibition of the growth of vessels, especially capillaries, into avascular resting tumors, with the result that there is no net tumor growth owing to the balance that is achieved between apoptosis and proliferation; 2) Prevention of the migration of tumor cells owing to the absence of blood flow to and from tumors; and 3) Inhibition of endothelial cell proliferation, thus avoiding the paracrine growth-stimulating effect exerted on the surrounding tissue by the endothelial cells normally lining the vessels.

Compounds of the formula I, in regard of their ability to inhibit KDR and thus to modulate angiogenesis, are especially appropriate for the therapy of diseases related to VEGF receptor tyrosine kinase overexpression. Among these diseases, especially (e.g. ischemic) retinopathies, (e.g. age related) macula degeneration, psoriasis, obesity, haemangioblastoma, haemangioma, inflammatory diseases, such as rheumatoid or rheumatic inflammatory diseases, especially arthritis, such as rheumatoid arthritis, or other chronic inflammatory disorders, such as chronic asthma, arterial or post-transplantational atherosclerosis, endometriosis, and especially neoplastic diseases, for example so-called solid tumors (especially cancers of the gastrointestinal tract, the pancreas, breast, stomach, cervix, bladder, kidney, prostate, ovaries, endometrium, lung, brain, melanoma, Kaposi's sarcoma, squamous cell carcinoma of head and neck, malignant pleural mesotherioma, lymphoma or multiple myeloma) and further liquid tumors (e.g. leukemias) are especially important.

The present invention can also be used to prevent or treat diseases that are triggered by persistent angiogenesis, such as restenosis, e.g., stent-induced restenosis; Crohn's disease; Hodgkin's disease; eye diseases, such as diabetic retinopathy and neovascular glaucoma; renal diseases, such as glomerulonephritis; diabetic nephropathy; inflammatory bowel disease; malignant nephrosclerosis; thrombotic microangiopathic syndromes; (e.g. chronic) transplant rejections and glomerulopathy; fibrotic diseases, such as cirrhosis of the liver; mesangial cell-proliferative diseases; injuries of the nerve tissue; and for inhibiting the re-occlusion of vessels after balloon catheter treatment, for use in vascular prosthetics or after inserting mechanical devices for holding vessels open, such as, e.g., stents, as immunosuppressants, as an aid in scar-free wound healing, and for treating age spots and contact dermatitis.

Preferably, the compounds of the formula I, or pharmaceutically acceptable salts thereof, are useful in the treatment of solid tumors as mentioned herein and/or of liquid tumors, e.g. leukemias, as mentioned herein.

Process of Manufacture

Compounds of formula I are prepared analogously to methods that, for other compounds, are in principle known in the art, preferably by reacting a boronic acid derivative of the formula II, 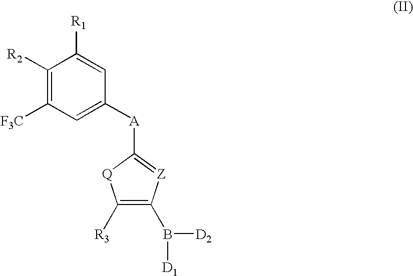
wherein D₁ and D₂ are hydroxy or substituted hydroxy, or together with the binding boron atom and two binding oxygen atoms form a ring of the formula IIA, 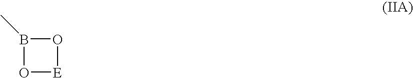
wherein E is alkylene, substituted alkylene, unsubstituted or substituted cycloalkylene,
unsubstituted or substituted bicycloalkylene or unsubstituted or substituted tricycloalkylene, with a coupling partner of the formula III,
   R₄-L (III)
   wherein R₄ is as defined above or below for a compound of the formula I and L is a leaving group;
   and, if desired, transforming a compound of formula I into a different compound of formula I, transforming a salt of an obtainable compound of formula I into the free compound or a different salt, and/or transforming an obtainable free compound of formula I into a salt thereof.

The reaction preferably takes place under customary conditions e.g. for the Suzuki-Miyaura cross-coupling (see e.g. Miyaura et al., Chem. Rev. 95, 2457 (1995)), in the presence of an appropriate (preferably water-free=absolute) solvent, for example an ether, such as ethylene glycol dimethyl ether or dioxane, a haydrocarbon, such as hexanes or toluene, or an alcohol, such as ethanol, or a mixture of any two or more thereof, in the presence of a catalyst, especially a noble metal complex catalyst, for example an iridium, a rhodium or preferably a palladium catalyst, such as tetrakis(triphenylphosphin)-palladium (Pd(PPh₃)₄) (which may also be formed in situ, e.g. from a palladium salt, such as palladium acetate, and the complex ligand, e.g. triphenylphosphin), preferably in the presence of a base, e.g. an acid addition salt of a metal, such as an alkali metal salt of an inorganic acid, e.g. a (e.g. sodium or potassium) phosphate or carbonate, or of a carbonic acid, e.g. a (e.g. sodium or potassium) lower alkanoate, such as acetate, at preferably elevated temperatures, e.g. between 25° C. and the reflux temperature, e.g. between 75 and 95° C. The reaction preferably takes place under an inert gas, such as nitrogen or argon.

If D₁ and D₂ each are substituted hydroxy, then substituted hydroxy is preferably alkyloxy, especially lower alkyloxy, aryloxy, especially phenyloxy with unsubstituted or substituted phenyl as defined above, or cycloalkyloxy wherein cycloalkyl is preferably C₃-C₈-cycloalkyl, such as cyclopentyl or cyclohexyl.

If (as is preferred) D₁ and D₂ together with the binding boron atom and oxygen atoms form a ring or the formula IIA shown above, then E preferably carries the two oxygen atoms bound to the boron atom on two different carbon atoms that are spatially nearby or neighbouring carbon atoms, e.g. in vicinal (“1,2-”) or in “1,3”-position (relatively to each other).

Alkylene is preferably an unbranched C₂-C₁₂—, preferably C₂-C₇alkylene moiety, e.g. ethylene, or propylene, in a broader aspect butylene, pentylene or hexylene, bound via two different carbon atoms as just described, preferably vicinal or in “1,3”-position. Substituted alkylene (which is preferred) is preferably an unbranched lower alkylene moiety as defined above which is substituted or unsubstituted by one or more, especially up to four, substituents preferably independently selected from lower alkyl, such as methyl or ethyl, e.g. in 1-methylethylene, 1,2-dimethylethylene, (preferably) 2,2-dimethylpropylene (neopentylene) or (especially preferred) 1,1,2,2-tetramethylethylene, or in a broader sense of the invention hydroxy, e.g. in 2-hydroxy-propylene, or hydroxy-lower alkyl, such as hydroxymethyl, e.g. in 1-hydroxymethyl-ethylene.

Unsubstituted or substituted cycloalkylene is preferably C₃-C₁₂—, more preferably C₃-C₈-cycloalkylene bound via two different carbon atoms as described for W, preferably vicinal or in “1,3”-position, such as cyclohexylene or cyclopentylene. Unsubstituted or substituted bicycloalkylene is preferably C₅-C₁₂-bicycloalkylene bound via two different carbon atoms as described for E, preferably vicinal or in “1,3”-position. An example is pinanylene (2,3-(2,6,6-trimethyl-bicyclo[3.1.1]heptane). Unsubstituted or substituted tricycloalkylene is preferably C₈-C₁₂-tricycloalkylene bound via two different carbon atoms as described for E, preferably vicinal or in “1,3”-position.

Unsubstituted or substituted cycloalkylene, unsubstituted or substituted bicycloalkylene or unsubstituted or substituted tricycloalkylene may be unsubstituted or substituted by one or more, especially up to three substituents independently selected from lower alkyl, such as methyl or ethyl, hydroxy, hydroxy-lower alkyl, such as methoxy, or mono- or oligosaccharidyl bound via an oxygen atom (“oligosaccharidyl” preferably comprising up to five saccharidyl moieties).

A leaving group L in a compound of the formula III is preferably halo, especially iodo, bromo (preferred) or chloro, or perfluoroalkylsulfonyloxy (e.g. —O—SO₂—(C_{f}F_{2f+1}), wherein f=1, 2 or 4).

In principle, manufacture of a compound of the formula I is alternatively also possible employing a compound of the formula II with a leaving group L instead of the group of the formula IIA given above and, as reaction partner, a compound of the formula III bearing a group of the formula IIA given above instead of the leaving group L. The reaction conditions then are analogous to those described for the reaction of the compounds of formula II and III given above.

Optional Reactions and Conversions

Compounds of the formula I may be converted into different compounds of the formula I. For example, lower alkoxycarbonyl substituents may be converted into carboxyl by saponification, nitro substituents may be hydrogenated to amino.

Salts of compounds of formula I having at least one salt-forming group may be prepared in a manner known per se. For example, salts of compounds of formula I having acid groups may be formed, for example, by treating the compounds with metal compounds, such as alkali metal salts of suitable organic carboxylic acids, e.g. the sodium salt of 2-ethylhexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of compounds of formula I are obtained in customary manner, e.g. by treating the compounds with an acid or a suitable anion exchange reagent. Internal salts of compounds of formula I containing acid and basic salt-forming groups, e.g. a free carboxy group and a free amino group, may be formed, e.g. by the neutralization of salts, such as acid addition salts, to the isoelectric point, e.g. with weak bases, or by treatment with ion exchangers.

A salt of a compound of the formula I can be converted in customary manner into the free compound; metal and ammonium salts can be converted, for example, by treatment with suitable acids, and acid addition salts, for example, by treatment with a suitable basic agent. In both cases, suitable ion exchangers may be used.

Intermediates and final products can be worked up and/or purified according to standard methods, e.g. using chromatographic methods, distribution methods, (re-) crystallization, and the like.

Starting Materials

The starting materials can, for example, preferably be prepared as follows:

A boronic acid derivative of the formula II is preferably prepared by reacting a compound of the formula IV, 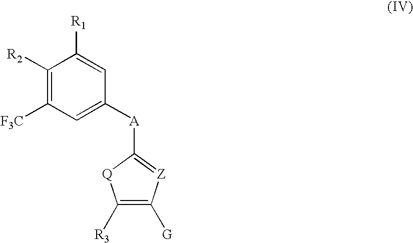
wherein R₁, R₂, R₃, A, Q and Z are as defined above for a compound of the formula I and G is a leaving group, especially as defined above for the leaving group L in a compound of the formula III, with a diboron compound of the formula VA or VB, 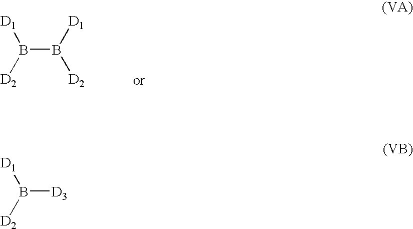
wherein D₁ and D₂ are as defined above for a compound of the formula II and D₃ is substituted hydroxy as defined above under formula II, under customary reaction conditions, that is in the presence of a in the presence of an appropriate (preferably water-free=absolute) solvent, for example an ether, such as ethylene glycol dimethyl ether, tetrahydrofurane or dioxane, a hydrocarbon, e.g. hexanes, or an alcohol, such as ethanol, or a mixture of any two or more thereof, in the presence of a noble metal complex catalyst, such as an iridium, rhodium or preferably palladium, e.g. preferably 1,1′-bis(diphenylphosphino)ferrocene-dichloro palladium (Pd(dppf)Cl₂), complex catalyst, and preferably in the presence of a base, e.g. an acid addition salt of a metal, such as an alkali metal salt of an inorganic acid, e.g. a (e.g. sodium or potassium) carbonate, or of a carbonic acid, e.g. a (e.g. sodium or potassium) lower alkanoate, such as acetate, at preferred temperatures e.g. between 20° C. and the reflux temperature, e.g. between 75 and the reflux temperature of the reaction mixture. The reaction preferably takes place under an inert gas, such as nitrogen or argon. Alternatively, the compound of the formula IV can first be lithiated, e.g. with n-butyllithium, and the resulting lithiated product then reacted with the compound of the formula VB under customary reaction conditions.

A starting material of the formula IV wherein R₁, R₂, R₃, Q and Z are as defined above or below for a compound of the formula I and G is a leaving group and A is —C(═O)—NH— (with the —NH-bound to the ring comprising Q and Z in formula I) is preferably manufactured by reacting a reactive derivative of a carbonic acid of the formula VI, 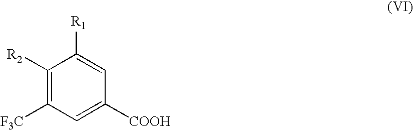
wherein R₁ and R₂ are as defined for a compound of the formula I, with an amino base of the formula VIII, 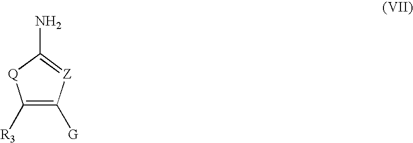
wherein Q, Z and R₃ and are as defined for a compound of the formula I and G is a leaving group as defined under formula IV, in an appropriate solvent, e.g. a nitrile, such as acetonitrile, preferably at a temperature from 0 to 50° C., e.g. from 20 to 40° C., preferably in the presence of a base, e.g. a tertiary nitrogen base, such as a tri-lower alkylamine, e.g. triethylamine. The active derivative is either converted in situ into a reactive derivative, e.g. by dissolving the compounds of formulae IV and V in a suitable solvent, for example N,N-dimethylformamide, N,N-dimethylacetamide, N-methyl-2-pyrrolidone, methylene chloride, or a mixture of two or more such solvents, and by the addition of a suitable base, for example triethylamine, diisopropyl-ethylamine (DIEA) or N-methylmorpholine and a suitable coupling agent that forms a preferred reactive derivative of the carbonic acid of formula III in situ, for example dicyclohexylcarbodiimide/1-hydroxybenzotriazole (DCC/HOBT); O-(1,2-dihydro-2-oxo-1-pyridyl)-N,N,N′,N′-tetramethyluronium tetrafluoroborate (TPTU); O-benzotriazol-1-yl)-N,N,N′,N′-tetramethyluronium tetrafluoroborate (TBTU); or 1-(3-dimethylaminopropyl)-3-ethylcarbodiimide hydrochloride (EDC). For review of other possible coupling agents, see e.g. Klauser; Bodansky, Synthesis 1972, 453-463. The reaction mixture is preferably stirred at a temperature of between approximately −20 and 50° C., especially between 0° C. and room temperature, to yield a compound of formula IV. Alternatively, the carbonic acid of the formula VI is used in the form of a reactive derivative, e.g. as the carbonic acid halide, such as chloride, as an anhydride with a carbonic acid, e.g. with a C₁-C₇-alkanoic acid, as an active ester, or in the form of an alkali metal salt, e.g. a sodium, lithium or potassium salt. In both cases, the reaction can preferably be carried out under an inert gas, e.g. nitrogen or argon.

A starting material of the formula IV wherein R₁, R₂, R₃, O and Z are as defined above or below for a compound of the formula I and G is a leaving group and A is —NH—C(═O)— (with the —C(═O)—bound to the ring comprising 0 and Z in formula I) can be synthesized from a reactive derivative (formed in situ or directly present, see the analogous reaction conditions using reactive derivatives of carbonic acids of the formula VI above) of a carbonic acid of the formula VIII, 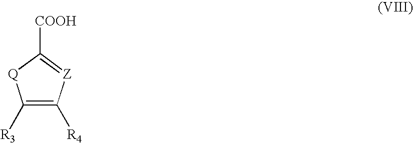
wherein R₃, Q and Z are as defined for a compound of the formula I and G is a leaving group as defined under formula IV, by reaction with an amino compound of the formula IX, 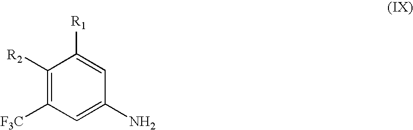
wherein R₁ and R₂ are as defined for a compound of the formula I, where the reaction conditions being used are analogous to those described herein for reaction of a compound of the formula VI and VII.

A compound of the formula III wherein L is a perfluoroalkanesulfonyloxy leaving group can be prepared, for example, by reacting a corresponding compound wherein instead of L a hydroxy group is present with a corresponding perfluoroalkanesulfonic anhydride, e.g. in an appropriate solvent, such as a halogenated hydrocarbon, e.g. dichloromethylene, in the presence of a (preferably tertiary nitrogen) base, such as a tri-lower alkylamine, e.g. triethylamine, a preferred temperatures from −10° C. to 50° C., e.g. from 0° C. to 25° C. A compound of the formula III wherein L is halo can, for example, be prepared by reacting a corresponding precursor compound wherein instead of L hydrogen is present, with a halogenating agent, e.g. N-bromosuccinimide in concentrated sulfuric acid/trifluoro acetic acid at preferred temperatures between 0 and 40° C., e.g. at room temperature.

Other starting materials, e.g. of the formula V, VI, VII, VII and IX, are known, can be obtained in analogy to methods that are known in the art and/or are commercially available, especially by or in analogy to methods given in the examples.

General Process Conditions

The following applies in general to all processes mentioned hereinbefore and hereinafter, while reaction conditions specifically mentioned above or below are preferred:

In any of the reactions mentioned hereinbefore and hereinafter, protecting groups may be used where appropriate or desired, even if this is not mentioned specifically, to protect functional groups that are not intended to take part in a given reaction, and they can be introduced and/or removed at appropriate or desired stages. Reactions comprising the use of protecting groups are therefore included as possible wherever reactions without specific mentioning of protection and/or deprotection are described in this specification.

Within the scope of this text, only a readily removable group that is not a constituent of the particular desired end product of formula I is designated a “protecting group”, unless the context indicates otherwise. The protection of functional groups by such protecting groups, the protecting groups themselves, and the reactions appropriate for their removal are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, in H.-D. Jakubke and H. Jesch-keit, “Aminosauren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide und Derivate” (Chemistry of Carbohydrates: Monosaccharides and Derivatives), Georg Thieme Verlag, Stuttgart 1974. A characteristic of protecting groups is that they can be removed readily (i.e. without the occurrence of undesired secondary reactions) for example by solvolysis, reduction, photolysis or alternatively under physiological conditions (e.g. by enzymatic cleavage).

All the above-mentioned process steps can be carried out under reaction conditions that are known per se, preferably those mentioned specifically, in the absence or, customarily, in the presence of solvents or diluents, preferably solvents or diluents that are inert towards the reagents used and dissolve them, in the absence or presence of catalysts, condensation or neutralizing agents, for example ion exchangers, such as cation exchangers, e.g. in the H⁺ form, depending on the nature of the reaction and/or of the reactants at reduced, normal or elevated temperature, for example in a temperature range of from about −100° C. to about 190° C., preferably from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofurane or dioxane, liquid aromatic hydrocarbons, such as benzene or toluene, alcohols, such as methanol, ethanol or 1- or 2-propanol, nitriles, such as acetonitrile, halogenated hydrocarbons, e.g. as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, or mixtures of these, for example aqueous solutions, unless otherwise indicated in the description of the processes. Such solvent mixtures may also be used in working up, for example by chromatography or partitioning.

The compounds, which term is in each case including the free compounds and/or their salts where salt-forming groups are present, may also be obtained in the form of hydrates, or their crystals may, for example, include the solvent used for crystallization, forming solvates. Different crystalline forms may be present.

The invention relates also to those forms of the process in which a compound obtainable as intermediate at any stage of the process is used as starting material and the remaining process steps are carried out, or in which a starting material is formed under the reaction conditions or is used in the form of a derivative, for example in protected form or in the form of a salt, or a compound obtainable by the process according to the invention is produced under the process conditions and processed further in situ. In the process of the present invention those starting materials are preferably used which result in compounds of formula I described as being preferred. Special preference is given to reaction conditions that are identical or analogous to those mentioned in the Examples.

### PREFERRED EMBODIMENTS ACCORDING TO THE INVENTION

In the following preferred embodiments, any one or more general expressions can be replaced by the corresponding more specific definitions provided above and below, thus yielding stronger preferred embodiments of the invention.

A preferred embodiment of the invention relates to a compound of the formula I wherein Q is —CH═CH— and R₁, R₂, R₃, R₄, R₅, A and Z are as defined for a compound of the formula I, or a (preferably pharmaceutically acceptable) salt thereof; or its use.

Another preferred embodiment of the invention relates to a compound of the formula I wherein A is —C(═O)—NH— (with the —NH— bound to the ring comprising Q and Z in formula I) and R₁, R₂, R₃, R₄, R₅, Q and Z are as defined for a compound of the formula I, or a (preferably pharmaceutically acceptable) salt thereof; or its use.

Another preferred embodiment relates to a compound of the formula I wherein one of R₁ and R₂ is hydrogen and the other is hydrogen or a moiety selected from the group consisting of 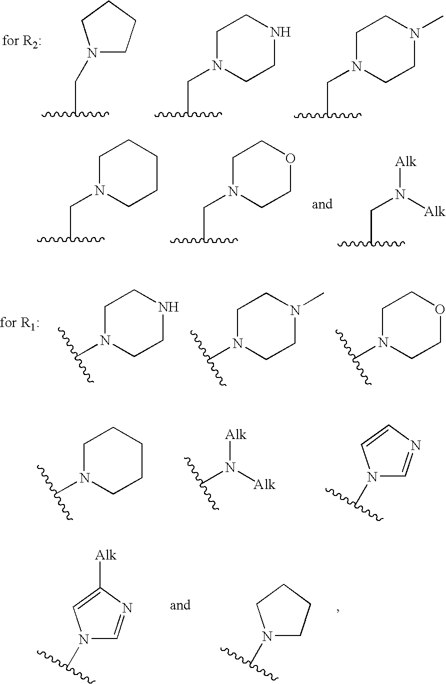
wherein “Alk” is alkyl, preferably lower alkyl, more preferably methyl or ethyl; and R₃, R₄, R₅, A, Q and Z are as defined above or below for a compound of the formula I, or a (preferably pharmaceutically acceptable) salt thereof.

The invention relates more preferably to a compound of the formula I, wherein each of R₁ and R₂ is hydrogen;
R₃ is C₁-C₇-alkyl, especially methyl;
R₄ is bicyclic heterocyclyl selected from the group consisting of 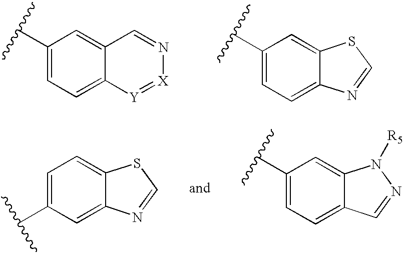
   wherein
X is CH, N or C—NH₂;
Y is CH or N;
   with the proviso that not both of X and Y are N simultaneously;
   and R₅ is hydrogen, C₁-C₇-alkyl or phenyl;
   (wherein R₄ is preferably

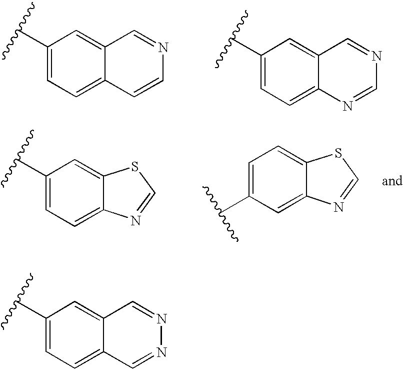

A is —C(═O)—NH— (with the —NH— bound to the ring comprising Q and Z in formula I) or —NH—C(═O)— (with the —C(═O)— bound to the ring comprising Q and Z in formula I);
Z is CH; and
Q is —CH═CH—;
   or a (preferably pharmaceutically acceptable) salt thereof where one or more salt-forming groups are present.

Another preferred embodiment of the invention relates to a compound of the formula I wherein R₄ is 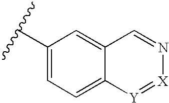
wherein
X is CH, N or C—NH₂;
Y is CH or N.

Another preferred embodiment of the invention relates to a compound of the formula I wherein R₄ is

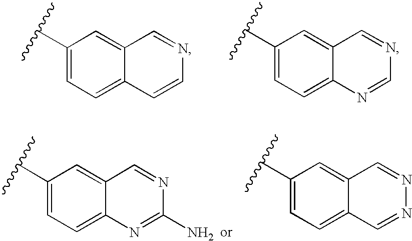

A preferred embodiment of the invention relates to the use (as defined above) of a compound of the formula I, or a pharmaceutically acceptable salt thereof; wherein Q is S and R₁, R₂, R₃, R₄, R₅, A and Z are as defined above or below for a compound of formula I.

Preferred is also the use (as defined above) of a compound of the formula I, or a pharmaceutically acceptable salt thereof, wherein A is NH—C(═O) (with the —C(═O)— bound to the ring comprising Q and Z in formula I) and R₁, R₂, R₃, R₄, R₅, Q and Z are as defined above or below for a compound of the formula I.

Very preferred is a method of treating a kinase dependent and/or proliferative disease comprising administering to an animal, especially a human, in need of such treatment a compound of formula I, where the disease to be treated is a proliferative disease, preferably a benign or especially malignant tumor, more preferably carcinoma of the brain, kidney, liver, adrenal gland, bladder, breast, stomach (especially gastric tumors), ovaries, colon, rectum, prostate, pancreas, lung, vagina, thyroid, sarcoma, glioblastomas, multiple myeloma or gastrointestinal cancer, especially colon carcinoma or colorectal adenoma, or a tumor of the neck and head, an epidermal hyperproliferation, especially psoriasis, prostate hyperplasia, a neoplasia, especially of epithelial character, preferably mammary carcinoma, or a leukemia. Also for the treatment of atherosclerosis, thrombosis, psoriasis, scleroderma and fibrosis, the compounds of the formula I are valuable. Other diseases or disorders in the treatment of which compounds of the formula I may be of use are atherosclerotic plaque rupture, osteoarthritis, chronic respiratory diseases (e.g. COPD, asthma), glomerulonephritis, neurodegenerative diseases (e.g. Alzheimer, Parkinson) and diabetic complications.

Most preferred is a compound of the formula I, or a (preferably pharmaceutically acceptable) salt thereof, as exemplified herein below under ‘Examples’, or its use as defined above.

Pharmaceutical Compositions

The invention relates also to pharmaceutical compositions comprising a compound of formula I, to their use in the therapeutic (in a broader aspect of the invention also prophylactic) treatment or a method of treatment of a kinase dependent disease, especially the preferred diseases mentioned above, to the compounds for said use and to pharmaceutical preparations and their manufacture, especially for said uses.

The present invention also relates to pro-drugs of a compound of formula I that convert in vivo to the compound of formula I as such. Any reference to a compound of formula I is therefore to be understood as referring also to the corresponding pro-drugs of the compound of formula I, as appropriate and expedient.

The pharmacologically acceptable compounds of the present invention may be present in or employed, for example, for the preparation of pharmaceutical compositions that comprise an effective amount of a compound of the formula I, or a pharmaceutically acceptable salt thereof, as active ingredient together or in admixture with one or more inorganic or organic, solid or liquid, pharmaceutically acceptable carriers (carrier materials).

The invention relates also to a pharmaceutical composition that is suitable for administration to a warm-blooded animal, especially a human (or to cells or cell lines derived from a warm-blooded animal, especially a human, e.g. lymphocytes), for the treatment (this, in a broader aspect of the invention, also including prevention of (=prophylaxis against)) a disease that responds to inhibition of kinase activity, comprising an amount of a compound of formula I or a pharmaceutically acceptable salt thereof, preferably which is effective for said inhibition, together with at least one pharmaceutically acceptable carrier.

The pharmaceutical compositions according to the invention are those for enteral, such as nasal, rectal or oral, or parenteral, such as intramuscular or intravenous, administration to warm-blooded animals (especially a human), that comprise an effective dose of the pharmacologically active ingredient, alone or together with a significant amount of a pharmaceutically acceptable carrier. The dose of the active ingredient depends on the species of warm-blooded animal, the body weight, the age and the individual condition, individual pharmacokinetic data, the disease to be treated and the mode of administration.

The invention relates also to a method of treatment for a disease that responds to inhibition of a kinase and/or a proliferative disease; which comprises administering a (against the mentioned disease) prophylactically or especially therapeutically effective amount of a compound of formula I according to the invention, or a pharmaceutically acceptable salt thereof, especially to a warm-blooded animal, for example a human, that, on account of one of the mentioned diseases, requires such treatment.

The dose of a compound of the formula I or a pharmaceutically acceptable salt thereof to be administered to warm-blooded animals, for example humans of approximately 70 kg body weight, preferably is from approximately 3 mg to approximately 10 g, more preferably from approximately 10 mg to approximately 1.5 g, most preferably from about 100 mg to about 1000 mg/person/day, divided preferably into 1-3 single doses which may, for example, be of the same size. Usually, children receive half of the adult dose.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, dragées, tablets or capsules.

The pharmaceutical compositions of the present invention are prepared in a manner known per se, for example by means of conventional dissolving, lyophilizing, mixing, granulating or confectioning processes.

Solutions of the active ingredient, and also suspensions, and especially isotonic aqueous solutions or suspensions, are preferably used, it being possible, for example in the case of lyophilized compositions that comprise the active ingredient alone or together with a carrier, for example mannitol, for such solutions or suspensions to be produced prior to use. The pharmaceutical compositions may be sterilized and/or may comprise excipients, for example preservatives, stabilizers, wetting and/or emulsifying agents, solubilizers, salts for regulating the osmotic pressure and/or buffers, and are prepared in a manner known per se, for example by means of conventional dissolving or lyophilizing processes. The said solutions or suspensions may comprise viscosity-increasing substances, such as sodium carboxymethylcellulose, carboxymethylcellulose, dextran, polyvinylpyrrolidone or gelatin.

Suspensions in oil comprise as the oil component the vegetable, synthetic or semi-synthetic oils customary for injection purposes. There may be mentioned as such especially liquid fatty acid esters that contain as the acid component a long-chained fatty acid having from 8-22, especially from 12-22, carbon atoms, for example lauric acid, tridecylic acid, myristic acid, pentadecylic acid, palmitic acid, margaric acid, stearic acid, arachidic acid, behenic acid or corresponding unsaturated acids, for example oleic acid, elaidic acid, erucic acid, brasidic acid or linoleic acid, if desired with the addition of antioxidants, for example vitamin E, α-carotene or 3,5-di-tert-butyl-4-hydroxytoluene. The alcohol component of those fatty acid esters has a maximum of 6 carbon atoms and is a mono- or poly-hydroxy, for example a mono-, di- or tri-hydroxy, alcohol, for example methanol, ethanol, propanol, butanol or pentanol or the isomers thereof, but especially glycol and glycerol. The following examples of fatty acid esters are therefore to be mentioned: ethyl oleate, isopropyl myristate, isopropyl palmitate, “Labrafil M 2375” (polyoxyethylene glycerol trioleate, Gattefossé, Paris), “Miglyol 812” (triglyceride of saturated fatty acids with a chain length of C8 to C12, Hüls AG, Germany), but especially vegetable oils, such as cottonseed oil, almond oil, olive oil, castor oil, sesame oil, soybean oil and groundnut oil.

The injection or infusion compositions are prepared in customary manner under sterile conditions; the same applies also to introducing the compositions into ampoules or vials and sealing the containers.

Pharmaceutical compositions for oral administration can be obtained by combining the active ingredient with solid carriers, if desired granulating a resulting mixture, and processing the mixture, if desired or necessary, after the addition of appropriate excipients, into tablets, dragée cores or capsules. It is also possible for them to be incorporated into plastics carriers that allow the active ingredients to diffuse or be released in measured amounts.

Suitable carriers are especially fillers, such as sugars, for example lactose, saccharose, mannitol or sorbitol, cellulose preparations and/or calcium phosphates, for example tricalcium phosphate or calcium hydrogen phosphate, and binders, such as starch pastes using for example corn, wheat, rice or potato starch, gelatin, tragacanth, methylcellulose, hydroxypropylmethylcellulose, sodium carboxymethylcellulose and/or polyvinylpyrrolidone, and/or, if desired, disintegrators, such as the above-mentioned starches, and/or carboxymethyl starch, crosslinked polyvinylpyrrolidone, agar, alginic acid or a salt thereof, such as sodium alginate. Excipients are especially flow conditioners and lubricants, for example silicic acid, talc, stearic acid or salts thereof, such as magnesium or calcium stearate, and/or polyethylene glycol. Dragée cores are provided with suitable, optionally enteric, coatings, there being used, inter alia, concentrated sugar solutions which may comprise gum arabic, talc, polyvinylpyrrolidone, polyethylene glycol and/or titanium dioxide, or coating solutions in suitable organic solvents, or, for the preparation of enteric coatings, solutions of suitable cellulose preparations, such as ethylcellulose phthalate or hydroxypropylmethylcellulose phthalate. Capsules are dry-filled capsules made of gelatin and soft sealed capsules made of gelatin and a plasticizer, such as glycerol or sorbitol. The dry-filled capsules may comprise the active ingredient in the form of granules, for example with fillers, such as lactose, binders, such as starches, and/or glidants, such as talc or magnesium stearate, and if desired with stabilizers. In soft capsules the active ingredient is preferably dissolved or suspended in suitable oily excipients, such as fatty oils, paraffin oil or liquid polyethylene glycols, it being possible also for stabilizers and/or antibacterial agents to be added. Dyes or pigments may be added to the tablets or dragée coatings or the capsule casings, for example for identification purposes or to indicate different doses of active ingredient.

Combinations

A compound of the formula I may also be used to advantage in combination with other antiproliferative agents. Such antiproliferative agents include, but are not limited to aromatase inhibitors; antiestrogens; topoisomerase I inhibitors; topoisomerase II inhibitors; microtubule active agents; alkylating agents; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; bisphosphonates; biological response modifiers; antiproliferative antibodies; heparanase inhibitors; inhibitors of Ras oncogenic isoforms; telomerase inhibitors; proteasome inhibitors; agents used in the treatment of hematologic malignancies; compounds which target, decrease or inhibit the activity of Flt-3; Hsp90 inhibitors; and temozolomide (TEMODAL®).

The term “aromatase inhibitor” as used herein relates to a compound which inhibits the estrogen production, i.e. the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane can be administered, e.g., in the form as it is marketed, e.g. under the trademark AROMASIN. Formestane can be administered, e.g., in the form as it is marketed, e.g. under the trademark LENTARON. Fadrozole can be administered, e.g., in the form as it is marketed, e.g. under the trademark AFEMA. Anastrozole can be administered, e.g., in the form as it is marketed, e.g. under the trademark ARIMIDEX. Letrozole can be administered, e.g., in the form as it is marketed, e.g. under the trademark FEMARA or FEMAR. Aminoglutethimide can be administered, e.g., in the form as it is marketed, e.g. under the trademark ORIMETEN. A combination of the invention comprising a chemotherapeutic agent which is an aromatase inhibitor is particularly useful for the treatment of hormone receptor positive tumors, e.g. breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen can be administered, e.g., in the form as it is marketed, e.g. under the trademark NOLVADEX. Raloxifene hydrochloride can be administered, e.g., in the form as it is marketed, e.g. under the trademark EVISTA. Fulvestrant can be formulated as disclosed in U.S. Pat. No. 4,659,516 or it can be administered, e.g., in the form as it is marketed, e.g. under the trademark FASLODEX. A combination of the invention comprising a chemotherapeutic agent which is an antiestrogen is particularly useful for the treatment of estrogen receptor positive tumors, e.g. breast tumors.

The term “anti-androgen” as used herein relates to any substance which is capable of inhibiting the biological effects of androgenic hormones and includes, but is not limited to, bicalutamide (CASODEX), which can be formulated, e.g. as disclosed in U.S. Pat. No. 4,636,505.

The term “gonadorelin agonist” as used herein includes, but is not limited to abarelix, goserelin and goserelin acetate. Goserelin is disclosed in U.S. Pat. No. 4,100,274 and can be administered, e.g., in the form as it is marketed, e.g. under the trademark ZOLADEX. Abarelix can be formulated, e.g. as disclosed in U.S. Pat. No. 5,843,901.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148 (compound A1 in WO99/17804). Irinotecan can be administered, e.g. in the form as it is marketed, e.g. under the trademark CAMPTOSAR. Topotecan can be administered, e.g., in the form as it is marketed, e.g. under the trademark HYCAMTIN.

The term “topoisomerase II inhibitor” as used herein includes, but is not limited to the anthracyclines such as doxorubicin (including liposomal formulation, e.g. CAELYX), daunorubicin, epirubicin, idarubicin and nemorubicin, the anthraquinones mitoxantrone and losoxantrone, and the podophillotoxines etoposide and teniposide. Etoposide can be administered, e.g. in the form as it is marketed, e.g. under the trademark ETOPOPHOS. Teniposide can be administered, e.g. in the form as it is marketed, e.g. under the trademark VM 26-BRISTOL. Doxorubicin can be administered, e.g. in the form as it is marketed, e.g. under the trademark ADRIBLASTIN or ADRIAMYCIN. Epirubicin can be administered, e.g. in the form as it is marketed, e.g. under the trademark FARMORUBICIN. Idarubicin can be administered, .e.g. in the form as it is marketed, e.g. under the trademark ZAVEDOS. Mitoxantrone can be administered, e.g. in the form as it is marketed, e.g. under the trademark NOVANTRON.

The term “microtubule active agent” relates to microtubule stabilizing, microtubule destabilizing agents and microtublin polymerization inhibitors including, but not limited to taxanes, e.g. paclitaxel and docetaxel, vinca alkaloids, e.g., vinblastine, especially vinblastine sulfate, vincristine especially vincristine sulfate, and vinorelbine, discodermolides, cochicine and epothilones and derivatives thereof, e.g. epothilone B or a derivative thereof. Paclitaxel may be administered e.g. in the form as it is marketed, e.g. TAXOL. Docetaxel can be administered, e.g., in the form as it is marketed, e.g. under the trademark TAXOTERE. Vinblastine sulfate can be administered, e.g., in the form as it is marketed, e.g. under the trademark VINBLASTIN R.P. Vincristine sulfate can be administered, e.g., in the form as it is marketed, e.g. under the trademark FARMISTIN. Discodermolide can be obtained, e.g., as disclosed in U.S. Pat. No. 5,010,099. Also included are Epothilone derivatives which are disclosed in WO 98/10121, U.S. Pat. No. 6,194,181, WO 98/25929, WO 98/08849, WO 99/43653, WO 98/22461 and WO 00/31247. Especially preferred are Epothilone A and/or B.

The term “alkylating agent” as used herein includes, but is not limited to, cyclophosphamide, ifosfamide, melphalan or nitrosourea (BCNU or Gliadel). Cyclophosphamide can be administered, e.g., in the form as it is marketed, e.g. under the trademark CYCLOSTIN. Ifosfamide can be administered, e.g., in the form as it is marketed, e.g. under the trademark HOLOXAN.

The term “histone deacetylase inhibitors” or “HDAC inhibitors” relates to compounds which inhibit the histone deacetylase and which possess antiproliferative activity. This includes compounds disclosed in WO 02/22577, especially N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(2-methyl-1H-indol-3-yl)-ethyl]-amino]methyl]phenyl]-2E-2-propenamide and pharmaceutically acceptable salts thereof. It further especially includes Suberoylanilide hydroxamic acid (SAHA).

The term “antineoplastic antimetabolite” includes, but is not limited to, 5-fluorouracil (5-FU); capecitabine; gemcitabine; DNA demethylating agents, such as 5-azacytidine and decitabine; methotrexate; edatrexate; and folic acid antagonists such as pemetrexed. Capecitabine can be administered, e.g., in the form as it is marketed, e.g. under the trademark XELODA. Gemcitabine can be administered, e.g., in the form as it is marketed, e.g. under the trademark GEMZAR. Also included is the monoclonal antibody trastuzumab which can be administered, e.g., in the form as it is marketed, e.g. under the trademark HERCEPTIN.

The term “platin compound” as used herein includes, but is not limited to, carboplatin, cis-platin, cisplatinum and oxaliplatin. Carboplatin can be administered, e.g., in the form as it is marketed, e.g. under the trademark CARBOPLAT. Oxaliplatin can be administered, e.g., in the form as it is marketed, e.g. under the trademark ELOXATIN.

The term “compounds targeting/decreasing a protein or lipid kinase activity and further anti-angiogenic compounds” as used herein includes, but is not limited to: protein tyrosine kinase and/or serine and/or threonine kinase inhibitors or lipid kinase inhibitors, e.g.:
a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, e.g. a N-phenyl-2-pyrimidine-amine derivative, e.g. imatinib, SU101, SU6668, and GFB-111;
b) compounds targeting, decreasing or inhibiting the activity of the fibroblast growth factor-receptors (FGFR);
c) compounds targeting, decreasing or inhibiting the activity of the insulin-like growth factor I receptor (IGF-R), especially compounds which inhibit the IGF-R, such as those compounds disclosed in WO 02/092599;
d) compounds targeting, decreasing or inhibiting the activity of the Trk receptor tyrosine kinase family;
e) compounds targeting, decreasing or inhibiting the activity of the Axl receptor tyrosine kinase family;
f) compounds targeting, decreasing or inhibiting the activity of the c-Met receptor;
g) compounds targeting, decreasing or inhibiting the activity of the c-Kit receptor tyrosine kinases—(part of the PDGFR family), such as compounds which target, decrease or inhibit the activity of the c-Kit receptor tyrosine kinase family, especially compounds which inhibit the c-Kit receptor, e.g. imatinib;
h) compounds targeting, decreasing or inhibiting the activity of members of the c-Abl family and their gene-fusion products (e.g. BCR-Abl kinase), such as compounds which target decrease or inhibit the activity of c-Abl family members and their gene fusion products, e.g. a N-phenyl-2-pyrimidine-amine derivative, e.g. imatinib; PD180970; AG957; NSC 680410; or PD173955 from ParkeDavis;
i) compounds targeting, decreasing or inhibiting the activity of members of the protein kinase C (PKC) and Raf family of serine/threonine kinases, members of the MEK, SRC, JAK, FAK, PDK and Ras/MAPK family members, or PI(3) kinase family, or of the PI(3)-kinase-related kinase family, and/or members of the cyclin-dependent kinase family (CDK) and are especially those staurosporine derivatives disclosed in U.S. Pat. No. 5,093,330, e.g. midostaurin; examples of further compounds include e.g. UCN-01, safingol, BAY 43-9006, Bryostatin 1, Perifosine; Ilmofosine; RO 318220 and RO 320432; GO 6976; Isis 3521; LY333531/LY379196; isochinoline compounds such as those disclosed in WO 00/09495; FTls; PD184352 or QAN697 (a P13K inhibitor);
j) compounds targeting, decreasing or inhibiting the activity of a protein-tyrosine kinase, such as imatinib mesylate (GLIVEC/GLEEVEC) or tyrphostin. A tyrphostin is preferably a low molecular weight (Mr<1500) compound, or a pharmaceutically acceptable salt thereof, especially a compound selected from the benzylidenemalonitrile class or the S-arylbenzenemalonirile or bisubstrate quinoline class of compounds, more especially any compound selected from the group consisting of Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); and
k) compounds targeting, decreasing or inhibiting the activity of the epidermal growth factor family of receptor tyrosine kinases (EGFR, ErbB2, ErbB3, ErbB4 as homo- or heterodimers), such as compounds which target, decrease or inhibit the activity of the epidermal growth factor receptor family are especially compounds, proteins or antibodies which inhibit members of the EGF receptor tyrosine kinase family, e.g. EGF receptor, ErbB2, ErbB3 and ErbB4 or bind to EGF or EGF related ligands, and are in particular those compounds, proteins or monoclonal antibodies generically and specifically disclosed in WO 97/02266, e.g. the compound of ex. 39, or in EP 0 564 409, WO 99/03854, EP 0520722, EP 0 566 226, EP 0 787 722, EP 0 837 063, U.S. Pat. No. 5,747,498, WO 98/10767, WO 97/30034, WO 97/49688, WO 97/38983 and, especially, WO 96/30347 (e.g. compound known as CP 358774), WO 96/33980 (e.g. compound ZD 1839) and WO 95/03283 (e.g. compound ZM105180); e.g. trastuzumab (HerpetinR), cetuximab, Iressa, erlotinib (Tarceva™), Cl-1033, EKB-569, GW-2016, E1.1, E2.4, E2.5, E6.2, E6.4, E2.11, E6.3 or E7.6.3, and 7H-pyrrolo-[2,3-d]pyrimidine derivatives which are disclosed in WO 03/013541.

Further anti-angiogenic compounds include compounds having another mechanism for their activity, e.g. unrelated to protein or lipid kinase inhibition e.g. thalidomide (THALOMID) and TNP-470.

Compounds which target, decrease or inhibit the activity of a protein or lipid phosphatase are e.g. inhibitors of phosphatase 1, phosphatase 2A, PTEN or CDC25, e.g. okadaic acid or a derivative thereof.

Compounds which induce cell differentiation processes are e.g. retinoic acid, α- γ- or δ-tocopherol or α- γ- or δ-tocotrienol.

The term “cyclooxygenase inhibitor” as used herein includes, but is not limited to, e.g. Cox-2 inhibitors, 5-alkyl substituted 2-arylaminophenylacetic acid and derivatives, such as celecoxib (CELEBREX), rofecoxib (VIOXX), etoricoxib, valdecoxib or a 5-alkyl-2-arylaminophenylacetic acid, e.g. 5-methyl-2-(2′-chloro-6′-fluoroanilino)phenyl acetic acid, lumiracoxib.

The term “mTOR inhibitors” relates to compounds which inhibit the mammalian target of rapamycin (mTOR) and which possess antiproliferative activity such as sirolimus (Rapamune®), everolimus (Certican™), CCl-779 and ABT578.

The term “bisphosphonates” as used herein includes, but is not limited to, etridonic, clodronic, tiludronic, pamidronic, alendronic, ibandronic, risedronic and zoledronic acid. “Etridonic acid” can be administered, e.g., in the form as it is marketed, e.g. under the trademark DIDRONEL. “Clodronic acid” can be administered, e.g., in the form as it is marketed, e.g. under the trademark BONEFOS. “Tiludronic acid” can be administered, e.g., in the form as it is marketed, e.g. under the trademark SKELID. “Pamidronic acid” can be administered, e.g. in the form as it is marketed, e.g. under the trademark AREDIA™. “Alendronic acid” can be administered, e.g., in the form as it is marketed, e.g. under the trademark FOSAMAX. “Ibandronic acid” can be administered, e.g., in the form as it is marketed, e.g. under the trademark BONDRANAT. “Risedronic acid” can be administered, e.g., in the form as it is marketed, e.g. under the trademark ACTONEL. “Zoledronic acid” can be administered, e.g. in the form as it is marketed, e.g. under the trademark ZOMETA.

The term “heparanase inhibitor” as used herein refers to compounds which target, decrease or inhibit heparin sulphate degradation. The term includes, but is not limited to, PI-88.

The term “biological response modifier” as used herein refers to a lymphokine or interferons, e.g. interferon γ.

The term “inhibitor of Ras oncogenic isoforms”, e.g. H-Ras, K-Ras, or N-Ras, as used herein refers to compounds which target, decrease or inhibit the oncogenic activity of Ras e.g. a “farnesyl transferase inhibitor”, e.g. L-744832, DK8G557 or R115777 (Zarnestra).

The term “telomerase inhibitor” as used herein refers to compounds which target, decrease or inhibit the activity of telomerase. Compounds which target, decrease or inhibit the activity of telomerase are especially compounds which inhibit the telomerase receptor, e.g. telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase are e.g. bengamide or a derivative thereof.

The term “proteasome inhibitor” as used herein refers to compounds which target, decrease or inhibit the activity of the proteasome. Compounds which target, decrease or inhibit the activity of the proteasome include e.g. PS-341 and MLN 341.

The term “matrix metalloproteinase inhibitor” or (“MMP inhibitor”) as used herein includes, but is not limited to collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “agents used in the treatment of hematologic malignancies” as used herein includes, but is not limited to FMS-like tyrosine kinase inhibitors e.g. compounds targeting, decreasing or inhibiting the activity of Flt-3; interferon, 1-b-D-arabinofuransylcytosine (ara-c) and bisulfan; and ALK inhibitors e.g. compounds which target, decrease or inhibit anaplastic lymphoma kinase.

The term “compounds which target, decrease or inhibit the activity of Flt-3” are especially compounds, proteins or antibodies which inhibit Flt-3, e.g. PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteasome pathway. Compounds targeting, decreasing or inhibiting the intrinsic ATPase activity of HSP90 are especially compounds, proteins or antibodies which inhibit the ATPase activity of HSP90 e.g., 17-allylamino,17-demethoxygeldanamycin (17AAG), a geldanamycin derivative; other geldanamycin related compounds; radicicol and HDAC inhibitors.

The term “antiproliferative antibodies” as used herein includes, but is not limited to trastuzumab (Herceptin™), Trastuzumab-DM1, bevacizumab (Avastin™), rituximab (Rituxan®), PRO64553 (anti-CD40) and 2C4 Antibody. By antibodies is meant e.g. intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of formula I can be used in combination with standard leukemia therapies, especially in combination with therapies used for the treatment of AML. In particular, compounds of formula I can be administered in combination with e.g. farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

The structure of the active agents identified by code nos., generic or trade names may be taken from the actual edition of the standard compendium “The Merck Index” or from databases, e.g. Patents International (e.g. IMS World Publications).

The above-mentioned compounds, which can be used in combination with a compound of the formula I, can be prepared and administered as described in the art such as in the documents cited above.

A compound of the formula I may also be used to advantage in combination with known therapeutic processes, e.g., the administration of hormones or especially radiation.

A compound of formula I may in particular be used as a radiosensitizer, especially for the treatment of tumors which exhibit poor sensitivity to radiotherapy.

By “combination”, there is meant either a fixed combination in one dosage unit form, or a kit of parts for the combined administration where a compound of the formula I and a combination partner may be administered independently at the same time or separately within time intervals that especially allow that the combination partners show a cooperative, e.g. synergistic, effect, or any combination thereof.

### EXAMPLES

The following examples serve to illustrate the invention without limiting its scope:

Ratios of solvents, e.g., in eluents or solvent mixtures, are given in volume by volume (v/v) or in volume percent. Temperatures are measured in degrees Celsius. Unless otherwise indicated, the reactions take place at RT. The R_{f} values which indicate the ratio of the distance moved by each substance to the distance moved by the eluent front are determined on silica gel thin-layer plates (Merck, Darmstadt, Germany) by thin-layer chromatography using the respective named solvent systems.

The analytical HPLC conditions where HPLC is mentioned are as follows:

| | |
|---|---|
| Column: | (70 × 4.0 mm) HPLC column CC 70/4 |
| | Nucleosil 100-3 C18 (3 μm mean particle |
| | size, with silica gel covalently |
| | derivatized with octadecylsilanes, Macherey & |
| | Nagel, Düren, Germany). Detection by |
| | UV absorption at 215 nm. The retention |
| | times (t_{R}) are given in minutes. Flow rate: 1 ml/min. |
| Gradient: | 20% → 100% a) in b) for 5 min + 1 min 100% a). |
| | a): Acetonitrile + 0.1% TFA; b): |
| | water + 0.1% TFA. |

Other HPLC Conditions:

HPLC(GRAD3):

| | |
|---|---|
| Column: | (250 × 4.6 mm) packed with reversed-phase |
| | material C18-Nucleosil (5 μm mean |
| | particle size, with silica gel covalently |
| | derivatized with octadecylsilanes, Macherey |
| | & Nagel, Düren, Germany). Detection by UV |
| | absorption at 215 nm. The retention |
| | times (t_{R}) are given in minutes. Flow rate: 1 ml/min. |
| Gradient: | 5% → 40% a) in b) for 7.5 min + 7 min 40% a). |
| | a): Acetonitrile + 0.1% TFA; b): |
| | water + 0.1% TFA. |

The short forms and abbreviations used have the following definitions:

| | |
|---|---|
| conc. | concentrated |
| DMF | N,N-dimethylformamide |
| MS-ES | mass spectroscopy (electron spray) |
| h | hour(s) |
| Me | methyl |
| min | minute(s) |
| mL | milliliter(s) |
| m.p. | melting point |
| RT | room temperature |
| TFA | trifluoroacetic acid |
| THF | tetrahydrofuran (distilled over Na/benzophenone) |
| TLC | thin-layer chromatography |
| t_{R} | retention times |

### Example 1

### N-(3-Isoquinolin-7-yl-4-methyl-phenyl)-3-trifluoromethyl-benzamide

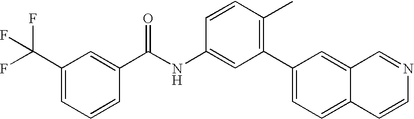

To a solution of N-[4-methyl-3-(4,4,5,5-tetramethyl-[1,3,2]dioxaborolan-2-yl)-phenyl]-3-tri-fluoromethyl-benzamide (1.74 g, 4.3 mmol) and trifluoro-methanesulfonic acid isoquinolin-7-yl ester (1.081 g, 3.9 mmol) in 28 mL of dry dioxane, 1.23 g (5.79 mmol) potassium phosphate are added and the solution is degassed by bubbling a slow stream of nitrogen through the suspension during 15 min. After the addition of 0.232 g (0.33 mmol) tetrakis-(triphenylphosphin)palladium the mixture is heated for 10 h to 90° C. The same amount of catalyst and potassium phosphate is added again, and the mixture is then stirred for 17 h at 90° C. The reaction mixture is cooled, filtered through Hyflo Super Cel® (Fluka, Buchs, Switzerland) and the residue washed with dioxane. The combined dioxane solutions are evaporated and the brown residue is purified by chromatography using a 120 g silica gel column on a Combi-Flash Companion™ (Isco Inc.) apparatus. A gradient of tert-butyl-methylether/hexane 1:1 to 4:1 is used. Pure fractions are pooled and evaporated to give the title compound as a pink foam; R_{f} (tert-butyl-methylether)=0.32; HPLC t_{R}=3.24 min; MS-ES+: (M+H)+=407.
Step 1.1: N-[4-Methyl-3-(4,4,5,5-tetramethyl-[1,3,2]dioxaborolan-2-yl)-phenyl]-3-trifluoromethyl benzamide

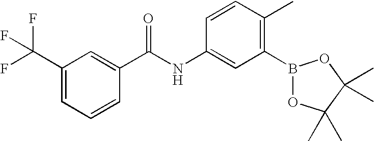

Nitrogen is bubbled through a mixture of 5.0 g (14 mmol) N-(3-bromo-4-methyl-phenyl)-3-trifluoromethyl-benzamide and 3.42 g (34.5 mmol) potassium acetate in 50 mL of THF for about 20 minutes. After the addition of 4.06 mg (16 mmol) bis-(pinacolato)-diboron, 6 mol-% of 1,1′-bis(diphenylphospino)ferrocene-palladium dichloride (700 mg, 0.8 mmol) is added and the resulting mixture heated under reflux for 18 h. The reaction mixture is then cooled to RT and diluted with ethyl acetate. After washing the mixture with conc. Sodium chloride solution, the ethyl acetate phase is dried with sodium sulphate and evaporated. The crude product is purified by flash chromatography using dichloromethane as solvent. The title compound is obtained as a colourless solid; m.p. 148-152° C.; R_{f} (dichloromethane)=0.36; HPLC t_{R}=4.82 min; MS-ES+: (M+H)+=406.
Step 1.2: N-(3-Bromo-4-methyl-phenyl)-3-trifluoromethyl-benzamide

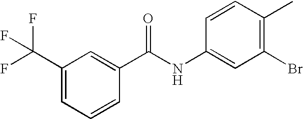

A solution of 5.8 mL (39 mmol) 3-trifluoromethyl-benzoyl chloride in 80 mL acetonitrile is treated drop-wise and at RT with 12.2 mL (78 mmol) triethylamine, followed by 7.8 g (42.9 mmol) 3-bromo-4-methyl-aniline. During the slow addition of the 3-trifluoromethyl-aniline, the temperature rises to about 30° C. The mixture is stirred at room temperature for 10 h and then cooled to 0° C. Water is added (100 mL) and the resulting precipitate filtered off, washed with water and dried. The solid is suspended in hexane stirred for a few min, filtered and dried again to give the title compound as a colourless solid; m.p. 153-155° C.; HPLC t_{R}=4.54 min.
Step 1.3: Trifluoro-methanesulfonic acid isoquinolin-7-yl ester

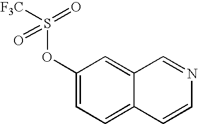

A solution of 5.8 g (0.04 mol) 7-hydroxyquinoline and 6.68 mL (0.048 mol) triethylamine in 100 mL of dichloromethane is cooled in an ice bath and treated dropwise over 30 min with 7.26 mL (0.044 mol) trifluoro-sulfonic acid anhydride. After complete addition, the cooling bath is removed and the dark mixture stirred for 1.5 h at RT. The reaction mixture is poured into 100 mL of ice-water and the bi-phasic mixture filtered through Hyflo Super Cel® (filtering aid based on diatomaceous earth; obtainable from Fluka, Buchs, Switzerland). The organic layer is separated and washed with 50 mL 10% citric acid, 50 mL of brine, dried with sodium sulphate and evaporated to leave a brown resin. This is purified by flash chromatography using dichloromethane/ethyl acetate 100:2.5 to 100:5. Pure fractions are pooled and evaporated to give an orange oil. HPLC t_{R}=2.35 min; R_{f} (tert-butyl-methylether)=0.38; MS-ES+: (M+H)+=278.

### Example 2

### N-(4-Methyl-3-quinazolin-6-yl-phenyl)-3-trifluoromethyl-benzamide

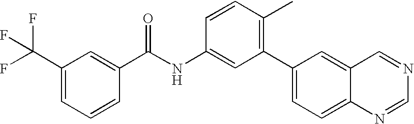

A mixture of N-[4-methyl-3-(4,4,5,5-tetramethyl-[1,3,2]dioxaborolan-2-yl)-phenyl]-3-tri-fluoromethyl-benzamide (0.456 g, 1.125 mmol) and 6-bromo-quinazoline (0.157 g, 0.75 mmol) in 3 mL of toluene and 0.375 mL of ethanol is treated with 0.75 mL of a 2 molar solution of sodium carbonate and the resulting mixture is degassed by bubbling nitrogen through the mixture for 5 min. After the addition of palladium acetate (0.0075 g, 0.034 mmol) and triphenylphosphine (0.0293 g, 0.117 mmol), the mixture is stirred at 90° C. for 2 h. The same amount of palladium acetate and triphenylphosphine is added again and the mixture stirred for 6 h at 90° C. The reaction mixture is cooled and added to 10 mL ethyl acetate and 4 mL of water. The bi-phasic mixture is filtered through Hyflo Super Cele (Fluka, Buchs, Switzerland), the organic layer separated, dried with sodium sulphate and evaporated to leave a brown resin. The crude product is purified by chromatography using a 40 g silica gel column on a Combi-Flash Companion™ (Isco Inc.) apparatus. A gradient of dichloromethane/methanol 100:1 to 100:15 is used. Enriched fractions are re-chromatographed on the same system using a 40 g silica gel column and tert-butyl-methylether as solvent. Pure fractions are pooled and evaporated to give the title compound as a tan foam; R_{f} (dichloromethane/ethanol 9:1)=0.56; HPLC t_{R}=3.23 min; MS-ES+: (M+H)+=408.
Step 2.1: 6-Bromo-quinazoline

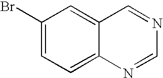

Trifluoroacetic acid (10 mL) is placed in a reaction vessel equipped with a thermometer and a mechanical stirrer. At 20° C., quinazoline (2.6 g, 0.020 mol) is added, followed by 3.4 mL of 96% sulphuric acid. N-Bromosuccinimide (4.8 g, 0.027 mol) is then added in 5 portions allowing 30 min in between the additions. After complete addition, the yellow mixture is stirred for 17 h at RT. The trifluoroacetic acid is removed on a rotary evaporator (rotavap) and the residue poored onto 20 g of crashed ice. The pH of the mixture is adjusted to ˜8-9 by the addition of 30% sodium hydroxide solution. The resulting suspension is diluted with 40 mL of ethyl acetate and filtered. The organic layer is separated and the aqueous phase extracted with 20 mL of ethyl acetate. The combined ethyl acetate extracts are dried with sodium sulphate and evaporated. Flash-chromatography of the residue using ethyl acetate/hexane 1:3 to 1:2 gives the title compound as colourless crystals. m.p. 155-156° C.; HPLC t_{R}=1.29 min; R_{f} (ethyl acetate/hexane 3:2)=0.36; MS-ES+: (M+H)+=210.9.

### Example 3

### 3-Isoquinolin-7-yl-4-methyl-N-(3-trifluoromethyl-phenyl)-benzamide

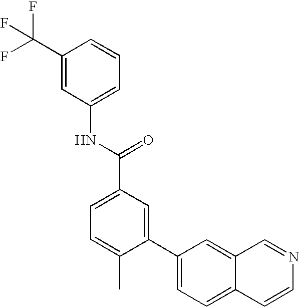

Using 4-methyl-3-(4,4,5,5-tetramethyl-[1,3,2]dioxaborolan-2-yl)-N-(3-trifluoromethyl-phenyl)-benzamide as different starting material, the same procedure as described in example 1 is used, except that no second addition of catalyst is required. The title compound is obtained as colourless solid; m.p. 189-191° C.; HPLC t_{R}=3.30 min; R_{f} (ethyl acetate/dichloromethane 1:4)=0.21; MS-ES+: (M+H)+=407.
Step 3.1: 4-Methyl-3-(4,4,5,5-tetramethyl-[1.3.2]dioxaborolan-2-yl)-N-(3-trifluoromethyl-phenyl)-benzamide

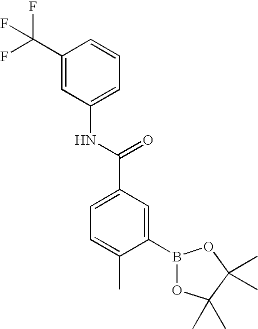

The same procedure is used as described in example 1, step 1.1 but starting with 3-bromo-4-methyl-N-(3-trifluoromethyl-phenyl)-benzamide. Reaction time is 8 h. The title compound is obtained as a tan solid; m.p. 157-159° C.; R_{f} (dichloromethane)=0.36; HPLC t_{R}=4.93 min; MS-ES+: (M+H)+=406.
Step 3.2: 3-Bromo-4-methyl-N-(3-trifluoromethyl-phenyl)-benzamide

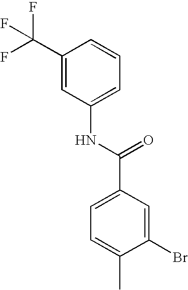

A solution of 14 g (60 mmol) 3-bromo-4-methyl-benzoyl chloride in 120 mL acetonitrile is treated drop-wise and at RT with 12.6 g (120 mmol) triethylamine, followed by 8.3 mL (66 mmol) 3-trifluoromethyl-aniline. During the slow addition of the 3-trifluoromethyl-aniline the temperature rises to about 35° C. The mixture is stirred at room temperature for 5 h and then diluted with ethyl acetate. The resulting mixture is washed sequentially with saturated sodium bicarbonate solution, 1 N hydrochloric acid and brine and then dried with sodium sulphate. Evaporation of the solvent leaves a brown oil which is crystallized from ether/petrol-ether to give the title compound as a colourless solid; m.p. 157-158° C.; HPLC t_{R}=4.63 min; R_{f} (dichloromethane)=0.75.

### Example 4

### 4-Methyl-3-quinazolin-6-yl-N-(3-trifluoromethyl-phenyl)-benzamide

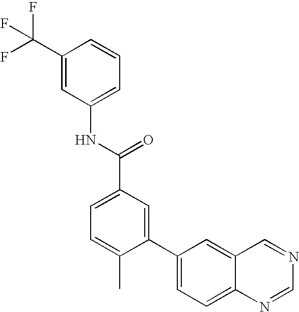

Using the title compound of example 3.1 as differing starting material, the same procedure as described in example 2 is used, except that no second addition of catalyst is required. The title compound is obtained as a colourless foam; HPLC t_{R}=3.31 min; R_{f} (tert.-butyl-methylether)=0.21; MS-ES+: (M+H)+=408.

### Example 5

### N-(3-Benzothiazol-6-yl-4-methyl-phenyl)-3-trifluoromethyl-benzamide

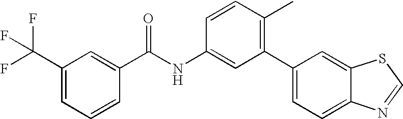

Using 6-bromo-benzothiazol as the differing starting material, the same procedure as described in example 2 is used, except that no second addition of catalyst is required. Reaction time 2 h, purification by flash chromatography. The title compound is obtained as a colourless solid; m.p. 94-96° C.; HPLC t_{R}=4.58 min; R_{f} (dichloromethane/ethanol 98:2)=0.3; MS-ES+: (M+H)+=413.

### Example 6

### 3-Benzothiazol-6-yl-4-methyl-N-(3-trifluoromethyl-phenyl)-benzamide

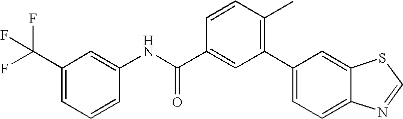

Using 6-bromo-benzothiazol and the title compound of example 3.1 as starting materials, the same procedure as described in example 2 is used, except that no second addition of catalyst is required. Reaction time 3 h. The title compound is obtained as a colourless solid; m.p. 102-104° C.; HPLC t_{R}=4.66 min; R_{f} (dichloromethane/ethanol 98:2)=0.3; MS-ES+: (M+H)+=413.

### Example 7

### N-(4-Methyl-3-phthalazin-6-yl-phenyl)-3-trifluoromethyl-benzamide

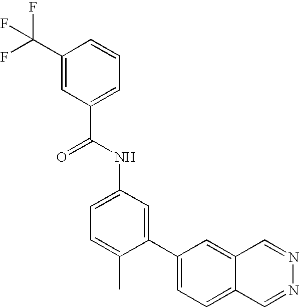

The same procedure as described in example 2 is used, except that no second addition of catalyst is required. Reaction time 3 h. The title compound is obtained as a colourless solid; m.p. 205-206° C.; HPLC t_{R}=3.34 min; MS-ES⁺: (M+H)⁺=408.

The starting material is prepared as follows:
Step 7.1: 6-Bromo-phthalazine

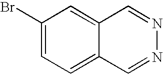

A solution of 1.0 g (4.7 mmol) 4-bromo-benzene-1,2-dicarbaldehyde in 4 mL of ethanol and 4 ml of dichloromethane is added dropwise over 40 min at 0° C. and under nitrogen to a solution of hydrazine hydrate (0.684 mL, 14.1 mmol) in 4.7 mL of ethanol. The resulting suspension is stirred 1 h at 0° C. and then the solvent is evaporated. The crystalline material is stirred with 20 mL of toluene and the solvent is evaporated again. This procedure is repeated with dichloromerthane. At the end the product is dried at 60° C. under vacuum for 8 h to give the title compound as colorless crystals: m.p. 140-143° C., HPLC t_{R}=1.49 min; ME-ES⁺: (M+H)⁺=210.9.
Step 7.2: 4-Bromo-benzene-1.2-dicarbaldehyde

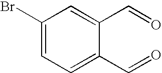

The title compound is synthesized by Swern oxidation of (4-bromo-2-hydroxymethyl-phenyl)-methanol following the procedure by O. Farooq, Synthesis 10, 1035-1037 (1994) and obtained as slightly yellow crystals: m.p. 97-100° C., MS-ES⁺: (M+H)⁺=210.9+212.9.
Step 7.3: 3-(4-Bromo-2-hydroxymethyl-phenyl)methanol

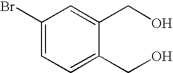

To a solution of 3 g (12.2 mmol) 4-bromo-phthalic acid in 24 mL of 1,2-dimethoxyethane, at 0° C. 1.394 g (36.8 mmol) of sodium borohydride are added in 10 portions. After stirring for 15 min, a solution of 4.61 mL (36.5 mmol) boron trifluoride etherate in 8 mL of 1,2-dimethoxyethane is added within 10 min. After stirring for 10 min at 0° C., the mixture is allowed to warm up to RT and stirring is continued for 2 h. The reaction mixture is then slowly added onto 40 g of crushed ice and the aqueous mixture is evaporated with ethyl acetate. The combined ethyl acetate extracts are washed with water and brine, dried with sodium sulfate and evaporated. The residual yellow oil (crude material) is purified by chromatography using a 120 g silica gel column on a Combi-Flash Companion™ (Isco Inc.) chromatography apparatus. A gradient of dichloromethane/ethyl acetate 0->50% ethyl acetate is used. The title compound is obtained as an oil which crystallizes on standing: m.p. 79-81° C., HPLC t_{R}=1.94 min, MS-ES⁺: (M+H)⁺=214+216.

### Example 8

### 4-Methyl-3-phthalazin-6-yl-N-(3-trifluoromethyl-phenyl)-benzamide

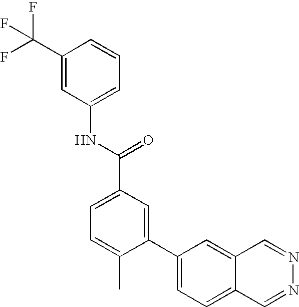

The same procedure as described in Example 7 is used. Title compound: m.p. 270-272° C.; HPLC t_{R}=3.43 min; R_{f} (dichloromethane/ethanol)=0.32; MS-ES⁺ (M+H)⁺=408.

### Example 9

### N-(3-Benzothiazol-5-yl-4-methyl-phenyl)-3-trifluoromethyl-benzamide

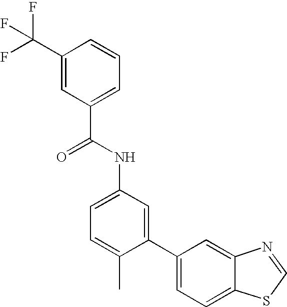

The same procedure as described in Example 2 is used starting with 5-bromo-benzothiazole. Reaction time total 4 h. The title compound is obtained as a colourless solid. M.p. 90-93° C., HPLC t_{R}=4.54 min; R_{f} (dichloromethane(ethanol)=0.30; MS-ES⁺: (M+H)⁺=413.

The starting material is prepared as follows:
Step 9.1: 5-Bromo-benzothiazole

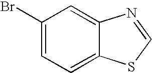

4-Amino-benztothiazole (3.0 g, 0.02 mol) in 18 mL of a 35% hydrobromic acid solution is diazotized at 0° C. by slow addition of a solution of 1.19 g (0,0195 mmol) sodium nitrite in 11 mL of water. After stirring for 1 h at 0° C. the brown solution is added dropwise to a dark solution of 3.3 g (0.023 mol) CuBr in 45 mL of a 35% hydrobromic acid solution at 0° C. The reaction mixture is stirred 0.5 h at 0° C., 2 h at RT and then 2 h at 90° C. The mixture is cooled to RT and pored into 20 g of crushed ice. Concentrated ammonia is added to the mixture to make it alkaline and then it is extracted with ethyl acetate. The organic layers are combined, washed with brine, dried with sodium sulfate and evaporated. The residue is purified by flash chromatography on silica gel using dichloromethane/petrol ether as eluent. The title compound is obtained as a solid: m.p. 104-106° C., HPLC t_{R}=3.44 min; R_{f} (dichloromethane/petrol ether)=0.30.
Step 9.2: 5-Amino-benzothiazole

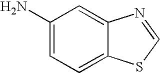

Purified 5-nitro-benzothiazole (7.2 g, 0.04 mol, see WO 98/23612, example 7A), dissolved in 160 mL of methanol and 160 mL of THF, is hydrogenated in the presence of 1.6 g Pd/C (10%; Engelhard 4505). The catalyst is filtered off, the filtrate concentrated and the residual oil purified by flash chromatography on silica gel using dichloromethanol/methanol 97:3 as eluent. The title compound is obtained as a colorless solid: m.p. 76-78° C., HPLC t_{R}=0.76 min; MS-ES⁺: (M+H)⁺=151; R_{f} (dichloromethane/methanol 97:3)=0.76.

### Example 10

### 3-Benzothiazol-5-yl-4-methyl-N-(3-trifluoromethylphenyl)benzamide

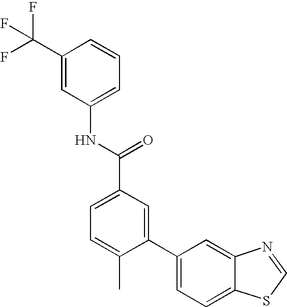

The same procedure as described in Example 9 is used. Title compound: m.p. 200-202° C., HPLC t_{R}=4.62 min; R_{f} (dichloromethane/ethanol 98:2)=0.30; MS-ES⁺: (M+H)⁺=413.

### Example 11

### N-(3-Isoquinolin-7-yl-4-methyl-phenyl)-4-(4-methyl-piperazin-1-ylmethyl)-3-trifluoromethyl-benzamide

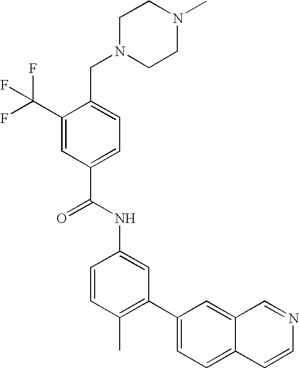

A solution of 0.162 g (0.584 mmol) trifluoro-methanesulfonic acid isoquinolin-7-yl ester (step 1.3) and 0.362 g (0.4897 mmol) 4-(4-methyl-piperazin-1-ylmethyl)-N-[4-methyl-3-(4,4,5,5-tetramethyl-[1,3,2]dioxaborolan-2-yl)-phenyl]-3-trifluoromethyl-benzamide in 4.2 mL dioxane is treated with 0.184 g (0.867 mmol) potassium phosphate. A slow stream of nitrogen is passed through the resulting suspension for 15 minutes, the mixture treated with 0.035 g (0.03 mmol) tetrakis(triphenylphosphine)palladium and then stirred at 90° C. for 4 h. Another 0.035 g (0.03 mmol) of the catalyst is added and stirring at 90° C. is continued for 15 h. The mixture is cooled, filtered and the filtrate evaporated. The residue is purified by chromatography using a 40 g silica gel column on a Combi-Flash Companion™ (Isco Inc.) apparatus. A gradient of dichloromethane/methanol (0→15% methanol) is used. Pure fractions are pooled and evaporated to give the title compound as a tan foam; R_{f} (dichloromethane/methanol 9:1)=0.23; HPLC t_{R}=2.47 min; MS-ES+: (M+H)+=519.
Step 11.1: 4-(4-Methyl-piperazin-1-ylmethyl)-N-[4-methyl-3-(4,4,5,5-tetramethyl-[1,3,2]dioxaborolan-2-yl)-phenyl]-3-trifluoromethyl-benzamide

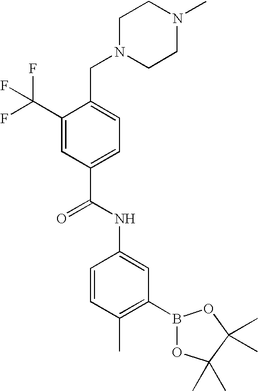

The title compound is synthesized following the same procedure as described in step 1.1 and using N-(3-bromo-4-methyl-phenyl)-4-(4-methyl-piperazin-1-ylmethyl)-3-trifluoromethyl-benzamide as starting material. The title compound as a tan foam; R_{f} (dichloromethane/methanol/conc. Ammonia 350:50:1)=0.88; HPLC t_{R}=3.70 min; MS-ES+: (M+H)+=518.
Step 11.2: N-(3-Bromo-4-methyl-phenyl)-4-(4-methyl-piperazin-1-ylmethyl)-3-trifluoromethyl-benzamide

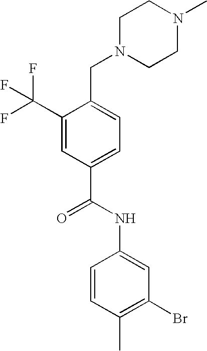

A solution of 4.51 g (0.01 mol) 4-bromomethyl-N-(3-bromo-4-methyl-phenyl)-3-trifluoromethyl-benzamide in 50 mL of acetone is cooled to 10° C. and treated with 2.76 g (0.02 mol) potassium carbonate and 1.33 mL (0.012 mol) 1-methylpiperazine. The mixture is stirred at rt for 4 h, filtered and the filtrate evaporated. The residue is dissolved in dichloromethane (50 mL) and washed with water, saturated sodium bicarbonate solution and water and dried with sodium sulphate. Evaporation of the solvent leads to pure title compound as a tan foam: R_{f} (ethyl acetate/methanol 8:2)=0.16; HPLC t_{R}=3.39 min; MS-ES+: (M+H)+=470, 472.
Step 11.3: 4-Bromomethyl-N-(3-bromo-4-methyl-phenyl)-3-trifluoromethyl-benzamide

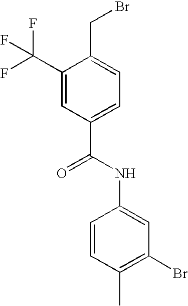

A solution containing 13.95 g (0.0493 mol) 4-bromomethyl-3-trifluoromethyl-benzoic acid, 9.17 g (0.0493 mol) 3-bromo-4-methylaniline and 7.56 g (0.0493 mol) 1-hydroxy-benzotriazole in 120 mL of THF is cooled to 0° C. and treated dropwise with a solution of 11.18 g (0.052 mol) N,N-dicyclohexylcarbodiimide in 40 mL of THF over 20 minutes at 0° C. After 45 minutes the cooling bath is removed and the mixture stirred for another hour at rt. The resulting suspension is filtered and the dicyclohexyl-urea washed with a small amount of THF. The filtrate is evaporated to dryness. The residue is purified by flash-chromatography on silica gel using ethyl acetate/hexanes first 2.5:100 then 15:100 as eluent. Pure fractions are pooled and evaporated to give crystalline title compound: m.p. 153-154° C.; R_{f} (ethyl acetate/hexanes 1:1)=0.63; HPLC t_{R}=4.72 min; MS-ES+: (M+H)+=450, 452.
Step 11.4: 4-Bromomethyl-3-trifluoromethyl-benzoic acid

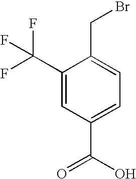

A suspension containing 16.33 g (0.08 mol) 4-methyl-3-(trifluoromethyl)-benzoic acid, 17.08 g (0.096 mol) N-bromosuccinimide and 0.96 g (0.003 mol) dibenzoyl-peroxide in 500 mL tetrachloromethane is heated under reflux and irradiated with a 125 W lamp for 1.5 h. The mixture is cooled to 10° C. filtered and the filtrate concentrated to about 50 mL. The solid is filtered off, washed with a small amount of cold tetrachloromethane and dried. The title compound was used without further purification: mp. 136-140° C.; HPLC t_{R}=3.40 min.

### Example 12

### 3-Isoquinolin-7-yl-4-methyl-N-[4-(4-methyl-piperazin-1-ylmethyl)-3-trifluoromethyl-phenyl]-benzamide

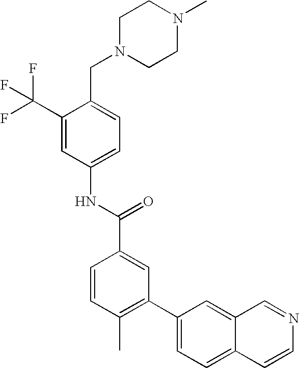

The title compound is synthesized following the same procedure as described in example 11 and using 4-methyl-N-[4-(4-methyl-piperazin-1-ylmethyl)-3-trifluoromethyl-phenyl]-3-(4,4,5,5-tetramethyl-[1,3,2]dioxaborolan-2-yl)-benzamide as starting material. The title compound as is obtained as a tan foam; R_{f} (dichloromethane/methanol 9:1)=0.10; HPLC t_{R}=2.34 min; MS-ES+: (M+H)+=519.
Step 12.1: 4-Methyl-N-[4-(4-methyl-piperazin-1-ylmethyl)-3-trifluoromethyl-phenyl]-3-(4,4,5,5-tetramethyl-[1,3,2]dioxaborolan-2-yl)-benzamide

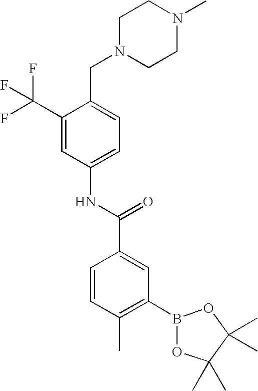

The title compound is synthesized following the same procedure as described in step 11.1 and using 3-bromo-4-methyl-N-[4-(4-methyl-piperazin-1-ylmethyl)-3-trifluoromethyl-phenyl]-benzamide as starting material. The title compound is obtained as a tan foam; R_{f} (dichloromethane/ethanol 9:1)=0.1; HPLC t_{R}=3.57 min; MS-ES+: (M+H)+=518.
Step 12.2: 3-Bromo-4-methyl-N-[4-(4-methyl-piperazin-1-ylmethyl)-3-trifluoromethyl-phenyl]-benzamide

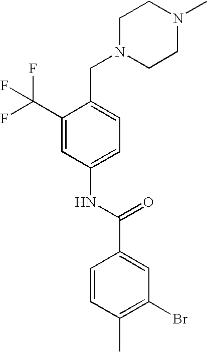

To a solution of 6.1 g (0.025 mol) 3-bromo-4-methylbenzoic acid chloride in 50 mL of acetonitrile are added at 10° C. 7 mL (0.05 mol) triethylamine followed by dropwise addition of a solution of 4-(4-methyl-piperazin-1-ylmethyl)-3-trifluoromethyl-phenylamine in 50 mL of acetonitrile (exothermic reaction). The brown suspension is stirred for 5 h at rt and is then allowed to stand over night. Ethyl acetate is added and the solution washed with saturated sodium bicarbonate solution and brine, dried with sodium sulphate and evaporated. Flash-chromatography on silica gel using dichloromethane/ethanol 93:7 containing 1% conc. ammonia gives pure title product: R_{f} (dichloromethane/ethanol 93:7 with 1% conc. ammonia)=0.4; HPLC t_{R}=3.14 min; MS-ES+: (M+H)+=470, 472.

### Example 13

### 4-(4-Methyl-piperazin-1-ylmethyl)-N-(4-methyl-3-quinazolin-6-yl-phenyl)-3-trifluoromethyl-benzamide

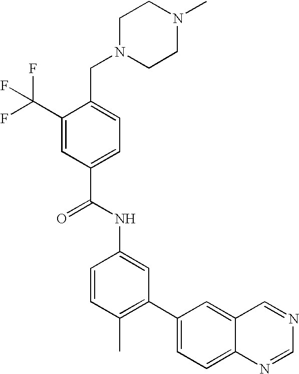

Nitrogen is passed for 10 minutes through a mixture containing 0.3 g (0.406 mmol) 4-(4-methyl-piperazin-1-ylmethyl)-N-[4-methyl-3-(4,4,5,5-tetramethyl-[1,3,2]dioxaborolan-2-yl)-phenyl]-3-trifluoromethyl-benzamide, 0.084 g (0.402 mmol) 6-bromo-quinazoline, 1.6 mL of toluene, 0.2 mL of ethanol and 0.4 mL of 2M sodium carbonate solution. The mixture is then treated under nitrogen with 4 mg (0.0178 mmol) palladium acetate and 15.6 mg (0.0595 mmol) triphenylphosphin and heated to 90° C. for 4 h. The dark mixture is treated with 5 mL of ethyl acetate and the organic phase is separated. 1.6 g of silica gel is added to the organic solution and the solvent is then removed. The crude product coated on the silica gel is purified by chromatography using a 40 g silica gel column on a Combi-Flash Companion™ (Isco Inc.) apparatus. A gradient of dichloromethane/ethanol (0→25% ethanol) is used. Pure fractions are pooled and evaporated to give the title compound as a tan foam; R_{f} (dichloromethane/ethanol 9:1)=0.07; HPLC t_{R}=2.48 min; MS-ES+: (M+H)+=520.

### Example 14

### 4-Methyl-N-[4-(4-methyl-piperazin-1-ylmethyl)-3-trifluoromethyl-phenyl]-3-quinazolin-6-yl-benzamide

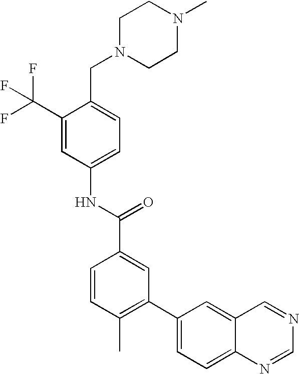

The title compound is synthesized following the same procedure as described in example 13 and using 4-methyl-N-[4-(4-methyl-piperazin-1-ylmethyl)-3-trifluoromethyl-phenyl]-3-(4,4,5,5-tetramethyl-[1,3,2]dioxaborolan-2-yl)-benzamide and 6-bromo-quinazoline as starting material. The title compound is obtained as a tan foam; R_{f} (dichloromethane/methanol 9:1)=0.18; HPLC t_{R}=2.36 min; MS-ES+: (M+H)+=520.

### Example 15

### N-(4-Methyl-3-phthalazin-6-yl-phenyl)-4-(4-methyl-piperazin-1-ylmethyl)-3-trifluoromethyl-benzamide

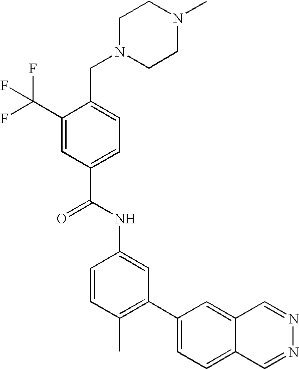

The title compound is synthesized following the same procedure as described in example 13 and using 4-(4-methyl-piperazin-1-ylmethyl)-N-[4-methyl-3-(4,4,5,5-tetramethyl-[1,3,2]dioxaborolan-2-yl)-phenyl]-3-trifluoromethyl-benzamide 6-bromophthalazine as starting material. The title compound is obtained as colourless crystals; m.p. 204-208° C.; HPLC t_{R}=2.53 min; MS-ES+: (M+H)+=520.

### Example 16

### 4-Methyl-N-[4-(4-methyl-piperazin-1-ylmethyl)-3-trifluoromethyl-phenyl]-3-phthalazin-6-yl-benzamide

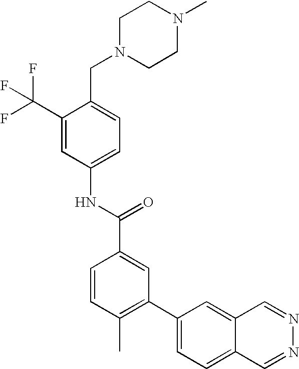

The title compound is synthesized following the same procedure as described in example 13 and using 4-methyl-N-[4-(4-methyl-piperazin-1-ylmethyl)-3-trifluoromethyl-phenyl]-3-(4,4,5,5-tetramethyl-[1,3,2]dioxaborolan-2-yl)-benzamide and 6-bromo-phthalazine as starting material. The title compound is obtained as a tan foam; R_{f} (dichloromethane/methanol 9:1)=0.18; HPLC t_{R}=2.38 min; MS-ES+: (M+H)+=520.

### Example 17

### N-(4-Methyl-3-phthalazin-6-yl-phenyl)-4-piperidin-1-ylmethyl-3-trifluoromethyl-benzamide

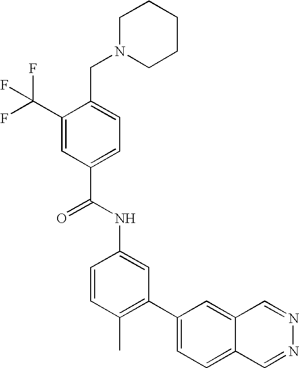

Nitrogen is bubbled through a mixture of 0.295 g (0.648 mmol) N-(3-bromo-4-methyl-phenyl)-4-piperidin-1-ylmethyl-3-trifluoromethyl-benzamide, 0.191 g (1.94 mmol) potassium acetate and 0.198 g (0.778 mmol) bis-(pinacolato)-diboron in 3.12 mL DMF for about 10 minutes. After the addition of 0.032 g (0.0391 mmol) 1,1′-bis(diphenylphospino)ferrocene-palladium dichloride the mixture is heated to 80° C. for 6 h. The N-[4-methyl-3-(4,4,5,5-tetramethyl-[1,3,2]dioxaborolan-2-yl)-phenyl]-4-piperidin-1-ylmethyl-3-trifluoromethyl-benzamide intermediate formed is not isolated. To the cooled dark suspension is added under nitrogen 6-bromophthalazine (0.1355 g, 0.648 mmol), caesium carbonate (0.316 g, 0.97 mmol) and 0.0225 mg (0.0195 mmol) tetrakis(triphenylphosphine)palladium. The dark mixture is heated to 80° C. for 15 h, cooled to rt and filtered. The solids are washed with DMF and the combined filtrates are evaporated under reduced pressure. The residue is partitioned between ethyl acetate and saturated sodium bicarbonate solution and the organic phase washed with brine, dried with sodium sulphate and evaporated. The crude product is purified by chromatography using a 40 g silica gel column on a Combi-Flash Companion™ (Isco Inc.) apparatus. A gradient of ethyl acetate/methanol (0→10% methanol) is used. Pure fractions are pooled and evaporated to give the title compound as tan crystals; m.p. 175-177° C.; R_{f} (ethyl acetate/methanol 9:1)=0.39; HPLC t_{R}=2.50 min; MS-ES+: (M+H)+=505.
Step 17.1: N-(3-Bromo-4-methyl-phenyl)-4-piperidin-1-ylmethyl-3-trifluoromethyl-benzamide

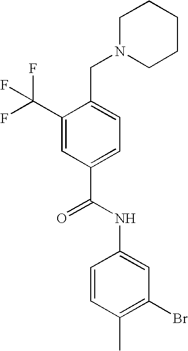

The title compound is synthesized following the same procedure as described in Step 11.2 and using piperidine as reagent. Tan foam: R1 (ethyl acetate)=0.71; HPLC t_{R}=3.51 min; MS-ES+: (M+H)+=455, 457.

### Example 18

### 4-Dimethylaminomethyl-N-(3-isoquinolin-7-yl-4-methyl-phenyl)-3-trifluoromethyl-benzamide

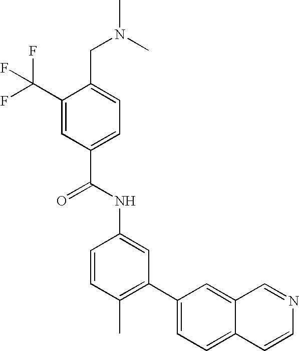

The title compound is synthesized following the same procedure as described in example 17 and using N-(3-Bromo-4-methyl-phenyl)-4-dimethylaminomethyl-3-trifluoromethyl-benzamide as starting material. colourless resin: R_{f} (ethyl acetate/methanol 9:1)=0.40; HPLC t_{R}=2.30 min; MS-ES+: (M+H)+=464.
Step 18.1: N-(3-Bromo-4-methyl-phenyl)-4-dimethylaminomethyl-3-trifluoromethyl-benzamide

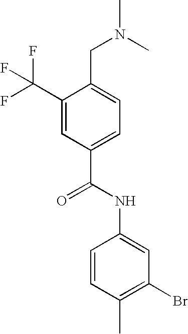

The title compound is synthesized following the same procedure as described in Step 11.2 and using dimethylamine hydrochloride as reagent. Yellowish crystals: m.p. 169-172° C.; R_{f} (ethyl acetate/methanol 9:1)=0.48; HPLC t_{R=}4.83 min; MS-ES+: (M+H)+=372, 374.

### Example 19

### 4-Dimethylaminomethyl-N-(4-methyl-3-phthalazin-6-yl-phenyl)-3-trifluoromethyl-benzamide

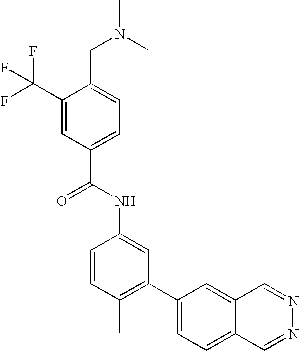

The title compound is synthesized following the same procedure as described in example 17 and using N-(3-bromo-4-methyl-phenyl)-4-dimethylaminomethyl-3-trifluoromethyl-benzamide and 6-bromophthalazine as starting material. Tan crystals: m.p. 240-241° C.; R_{f} (ethyl acetate/methanol 9:1)=0.20; HPLC t_{R}=2.24 min; MS-ES+: (M+H)+=465.

### Example 20

### N-(4-Methyl-3-phthalazin-6-yl-phenyl)-4-morpholin-4-ylmethyl-3-trifluoromethyl-benzamide

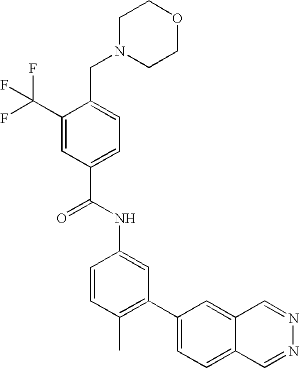

The title compound is synthesized following the same procedure as described in example 17 and using N-(3-bromo-4-methyl-phenyl)-4-morpholin-4-ylmethyl-3-trifluoromethyl-benzamide and 6-bromophthalazine as starting material. Tan crystals: m.p. 236-238° C.; R_{f} (ethyl acetate/methanol 92.5:7.5)=0.26; HPLC t_{R}=2.30 min; MS-ES+: (M+H)+=507.
Step 20.1: -(3-Bromo-4-methyl-phenyl)-4-morpholin-4-ylmethyl-3-trifluoromethyl-benzamide

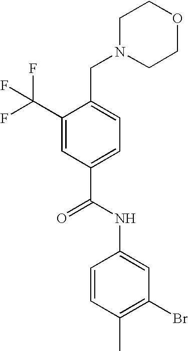

The title compound is synthesized following the same procedure as described in Step 11.2 and using morpholine as reagent. Colourless crystals: m.p. 160-162° C.; R_{f} (ethyl acetate/hexanes 1:1)=0.40; HPLC t_{R}=3.27 min; MS-ES+: (M+H)+=457, 459.

### Example 21

### N-(3-Isoquinolin-7-yl-4-methyl-phenyl)-4-morpholin-4-ylmethyl-3-trifluoromethyl-benzamide

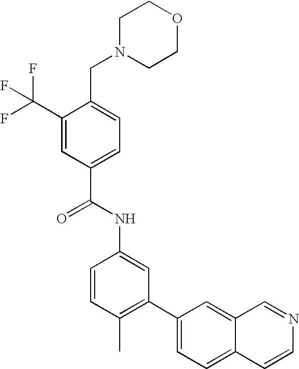

The title compound is synthesized following the same procedure as described in example 17 and using N-(3-bromo-4-methyl-phenyl)-4-morpholin-4-ylmethyl-3-trifluoromethyl-benzamide and trifluoro-methanesulfonic acid isoquinolin-7-yl ester as starting material. Colourless resin: R_{f} (ethyl acetate)=0.20; HPLC t_{R}=2.29 min; MS-ES+: (M+H)+=506.

### Example 22

### 4-Methyl-3-phthalazin-6-yl-N-(4-piperidin-1-ylmethyl-3-trifluoromethyl-phenyl)-benzamide

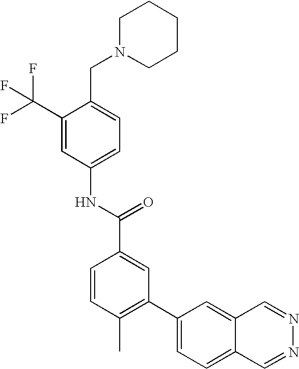

The title compound is synthesized following the same procedure as described in example 17 and using 3-bromo-4-methyl-N-(4-piperidin-1-ylmethyl-3-trifluoromethyl-phenyl)-benzamide and 6-bromophthalazine as starting material. Tan crystals: m.p. 247-249° C.; HPLC t_{R}=2.52 min; MS-ES+: (M+H)+=505.
Step 22.1: 3-Bromo-4-methyl-N-(4-piperidin-1-ylmethyl-3-trifluoromethyl-phenyl)-benzamide.

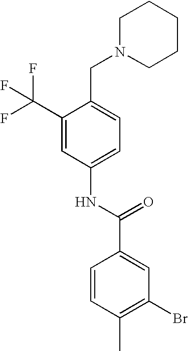

A solution of 0.5 g (1.295 mmol) 3-bromo-N-(4-formyl-3-trifluoromethyl-phenyl)-4-methyl-benzamide in 5 mL ethyl acetate is treated under nitrogen with 0.64 mL (6.48 mmol) piperidine and 0.0325 mg (0.13 mmol) pyridinium tosylate. The mixture is heated to 60° C. and sodium triacetoxyborohydride is added in small portions over 45 minutes. Stirring is continued at 60° C. for 10 minutes after which the thick suspension is allowed to stand at rt over night. At 10° C. the mixture is hydrolysed by the dropwise addition of 2 mL of water. The two layers are separated and the ethyl acetate phase washed with water and brine, dried with sodium sulphate and evaporated. The crude product is purified by chromatography using a 40 g silica gel column on a Combi-Flash Companion™ (Isco Inc.) apparatus. A gradient of ethyl acetate/hexanes (5→30% ethyl acetate) is used. Pure fractions are pooled and evaporated to give the title compound as light yellow crystals; m.p. 151-153° C.; R_{f} (ethyl acetate)=0.52; HPLC t_{R}=3.56 min; MS-ES+: (M+H)+=455, 457.
Step 22.2: 3-Bromo-N-(4-formyl-3-trifluoromethyl-phenyl)-4-methyl-benzamide

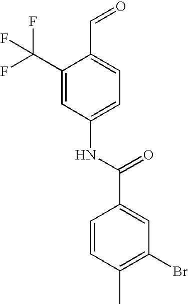

Crude 4-amino-2-trifluoromethyl-benzaldehyde (brown oil, ˜3 g, ˜0.016 mol) is dissolved in 15 mL of dichloromethane and treated at rt with triethylamine (2.465 mL, 0.0177 mol). To the dark solution is then slowly added a solution of 3.8 g (0.016 mol) 3-bromo-4-methylbenzoic acid chloride in 15 mL dichloromethane. After complete addition the mixture is allowed to stand over night at rt. The dichloromethane is evaporated and the residue is purified by chromatography using a 120 g silica gel column on a Combi-Flash Companion™ (Isco Inc.) apparatus. A gradient of ethyl acetate/hexanes (0→25% ethyl acetate) is used. Pure fractions are pooled and evaporated to give the title compound as light yellow crystals; m.p. 193.5-195° C.; R_{f} (ethyl acetate/hexanes 1:3)=0.34; HPLC t_{R}=4.75 min; MS-ES+: (M+H)+=386, 384.
Step 22.3: 4-Amino-2-trifluoromethyl-benzaldehyde

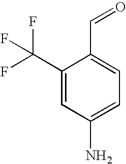

A solution of 3 g (0.0161 mol) 4-amino-2-trifluoromethyl-benzonitrile in 9 mL of dry THF is treated dropwise at rt and under nitrogen with 26.85 mL (0.0403 mol) of a 1.5 M diisobutyl-aluminum-hydride solution in toluene. During the addition the temperature is maintained at maximally 28° C. by appropriate cooling. After complete addition the brown solution is allowed to stand at rt over night. It is then added dropwise to a mixture of 4.4 mL of methanol and 39 mL of a saturated (˜3M) potassium sodium tartrate solution. During the hydrolysis the temperature is kept below 40° C. After stirring for 15 minutes ethyl acetate is added and the two layers separated. The ethyl acetate phase is washed with water and brine, dried with sodium sulphate and evaporated. The brown foam obtained consists of oligomeric forms of the aldehyde (imine formation) and is therefore re-dissolved in 10 mL of ethyl acetate and stirred efficiently for 10 minutes with 10 mL of 1 N HCl. Sodium hydroxide (1 N, 8.5 mL) is added and stirring is continued for 5 more minutes (at the end the solution has pH ˜9). The ethyl acetate is separated, washed with brine, dried with sodium sulphate and evaporated to give crude 4-amino-2-trifluoromethyl-benzaldehyde as a brown oil which is immediately used in the next step.

### Example 23

### 4-Methyl-N-(4-morpholin-4-ylmethyl-3-trifluoromethyl-phenyl)-3-phthalazin-6-yl-benzamide

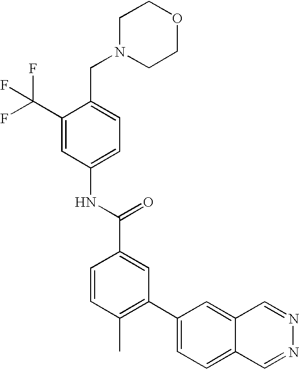

The title compound is synthesized following the same procedure as described in example 17 and using 3-bromo-4-methyl-N-(4-morpholin-4-ylmethyl-3-trifluoromethyl-phenyl)-benzamide and 6-bromophthalazine as starting material. Tan crystals: m.p. 284-287° C.; R_{f} (ethyl acetate/ethanol 95:5)=0.16; HPLC t_{R}=2.25 min; MS-ES+: (M+H)+=507.
Step 23.1: 3-Bromo-4-methyl-N-(4-morpholin-4-ylmethyl-3-trifluoromethyl-phenyl)-benzamide

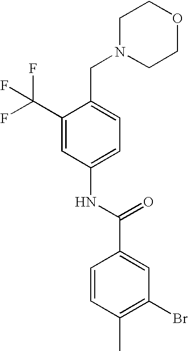

The title compound is synthesized following the same procedure as described in step 22.1 and using 3-bromo-N-(4-formyl-3-trifluoromethyl-phenyl)-4-methyl-benzamide and morpholine as starting material. Light yellow crystals: m.p. 147-151° C.; HPLC t_{R}=3.31 min; MS-ES+: (M+H)+=457, 459.

### Example 24

### N-(4-Dimethylaminomethyl-3-trifluoromethyl-Phenyl)-4-methyl-3-phthalazin-6-yl-benzamide

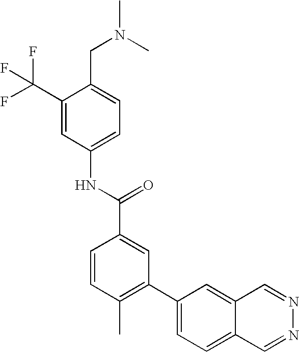

The title compound is synthesized following the same procedure as described in example 17 and using 3-bromo-N-(4-dimethylaminomethyl-3-trifluoromethyl-phenyl)-4-methyl-benzamide and 6-bromophthalazine as starting material. Colourless crystals: m.p. 251-254° C.; R_{f} (dichloromethane/methanol/conc. ammonia 90:10:1)=0.45; HPLC t_{R}=2.22 min; MS-ES+: (M+H)+=465.
Step 24.1: 3-Bromo-N-(4-dimethylaminomethyl-3-trifluoromethyl-phenyl)-4-methyl-benzamide

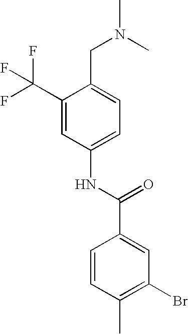

The title compound is synthesized following the same procedure as described in step 22.1 and using 3-bromo-N-(4-formyl-3-trifluoromethyl-phenyl)-4-methyl-benzamide and dimethylamine hydrochloride and triethylamine as starting material. Colourless crystals: m.p. 156-157° C.; HPLC t_{R}=3.24 min; MS-ES+: (M+H)+=415, 417.

### Example 25

### 4-Methyl-3-phthalazin-6-yl-N-(4-pyrrolidin-1-ylmethyl-3-trifluoromethyl-phenyl)-benzamide

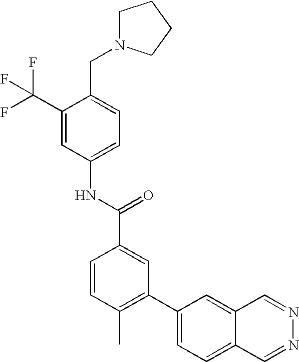

The title compound is synthesized following the same procedure as described in example 17 and using 3-bromo-4-methyl-N-(4-pyrrolidin-1-ylmethyl-3-trifluoromethyl-phenyl)-benzamide and 6-bromophthalazine as starting material. Colourless crystals: m.p. 246-250° C.; R_{f} (dichloromethane/methanol/conc. ammonia 90:10:1)=0.39; HPLC t_{R}=2.42 min; MS-ES+: (M+H)+=491.
Step 25.1: 3-Bromo-4-methyl-N-(4-pyrrolidin-1-ylmethyl-3-trifluoromethyl-phenyl)-benzamide

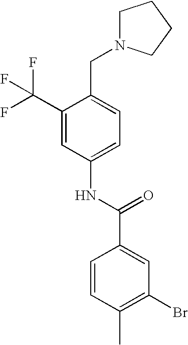

The title compound is synthesized following the same procedure as described in step 22.1 and using 3-bromo-N-(4-formyl-3-trifluoromethyl-phenyl)-4-methyl-benzamide and pyrrolidine as starting material. Colourless crystals: m.p. 168-170° C.; HPLC t_{R}=3.43 min; MS-ES+: (M+H)+=441, 443.

### Example 26

### N-(3-(2-Aminoquinazolin-6-yl)-4-methyl-phenyl)-4-(4-methyl-piperazin-1-ylmethyl)-3-trifluoromethyl-benzamide

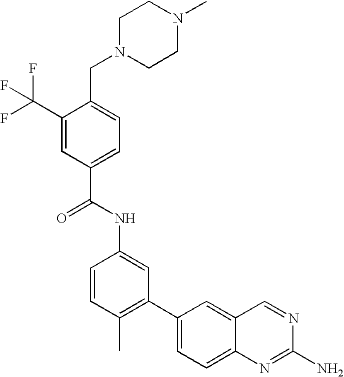

In a 50 mL sealed tube 0.400 g (1.70 mmol) 2-amino-6-bromo-quinazoline, 0.420 g (0.804 mmol) 4-(4-methyl-piperazin-1-ylmethyl)-N-[4-methyl-3-(4,4,5,5-tetramethyl-[1,3,2]dioxaborolan-2-yl)-phenyl]-3-trifluoromethyl-benzamide (step 11.1), and 0.160 g (0.226 mmol) bis(triphenylphosphine) palladium (II) chloride are added to a solution of 2 mL of 1 M aqueous sodium hydrogen carbonate, 5 mL toluene and 1 mL EtOH. After bubbling with nitrogen for 5 minutes, the reaction mixture is sealed and heated at 90° C. for 3 h. After cooling, the mixture is concentrated in vacuo and the resulting residue is purified by reverse phase HPLC using a Varian Prostar system equipped with a Waters xTerra column (50×100 mm) and a solvent gradient of 0.1% NH₃ in water/0.1% NH₃ in acetonitrile (0→100%). Pure fractions are pooled and evaporated to give 0.10 g (0.185 mmol) of the title compound as a light yellow solid; HPLC t_{R} (water/acetonitrile)=8.4 min; MS-ES+: (M+H)+=535.
Step 26.1: 2-Amino-6-bromo-quinazoline

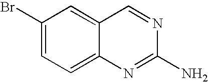

In a 250 mL reaction tube 9.30 g (45.4 mmol) 5-bromo-2-fluorobenzaldehyde and 12.40 g (68.1 mmol) guanidine carbonate are dissolved in 130 mL N, N-dimethylacetamide. After bubbling the solution with nitrogen for 1 h, the tube is sealed and heated at 140° C. for 3 h. After cooling the reaction is diluted with 50 mL of a saturated NaHCO₃ solution and 300 mL water and stirred for 0.5 h. The resulting precipitate is collected, washed first with 50 mL water followed by 50 mL ether, and air dried to give 4.0 g (17.7 mmol) of the titled compound: HPLC t_{R}=5.6 min; MS-ES+: (M+H)+=225.

### Example 27

### Soft Capsules

5000 soft gelatin capsules, each comprising as active ingredient 0.05 g of one of the compounds of formula I mentioned in any one of the preceding Examples, are prepared as follows:

| Composition: | | |
|---|---|---|
| Active ingredient | 250 | g |
| Lauroglycol | 2 | liters |

Preparation process: The pulverized active ingredient is suspended in Lauroglykol® (propylene glycol laurate, Gattefossé S. A., Saint Priest, France) and ground in a wet pulverizer to produce a particle size of about 1 to 3 μm. 0.419 g portions of the mixture are then introduced into soft gelatin capsules using a capsule-filling machine.

### Example 28

### Tablets Comprising Compounds of the Formula I

Tablets, comprising, as active ingredient, 100 mg of any one of the compounds of formula I of Examples 1 to 10 are prepared with the following composition, following standard procedures:

| Composition: | | |
|---|---|---|
| Active Ingredient | 100 | mg |
| crystalline lactose | 240 | mg |
| Avicel | 80 | mg |
| PVPPXL | 20 | mg |
| Aerosil | 2 | mg |
| magnesium stearate | 5 | mg |
| | 447 | mg |

Manufacture: The active ingredient is mixed with the carrier materials and compressed by means of a tabletting machine (Korsch EKO, Stempeldurchmesser 10 mm).

Avicel® is microcrystalline cellulose (FMC, Philadelphia, USA). PVPPXL is polyvinyl-polypyrrolidone, cross-linked (BASF, Germany). Aerosil® is silicum dioxide (Degussa, Germany).

1. A compound of the formula I, 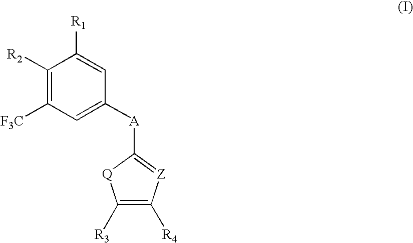
wherein
R₁ is hydrogen or —N(R₆R₇) wherein each of R₆ and R₇ is alkyl or R₆ and R₇, together with the nitrogen to which they are bound, form a 5- to 7-membered heterocyclic ring, where the additional ring atoms are selected from carbon and 0, 1 or 2 heteroatoms selected from nitrogen, oxygen and sulfur and which ring is unsubstituted or, if a further nitrogen ring atom is present, unsubstituted or substituted by alkyl at that nitrogen;
R₂ is hydrogen or —CH₂—N(R₆R₇) wherein each of R₆ and R₇ is alkyl or R₆ and R₇, together with the nitrogen to which they are bound, form a 5- to 7-membered heterocyclic ring, where the additional ring atoms are selected from carbon and 0, 1 or 2 heteroatoms selected from nitrogen, oxygen and sulfur and which ring is unsubstituted or, if a further nitrogen ring atom is present, unsubstituted or substituted by alkyl at that nitrogen;
with the proviso that at least one of R₁ and R₂ is hydrogen;
R₃ is halo or C₁-C₇-alkyl;
R₄ is bicyclic heterocyclyl selected from the group consisting of 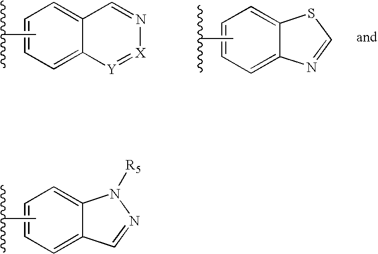
wherein
X is CH, N or C—NH₂;
Y is CH or N;
with the proviso that not both of X and Y are N simultaneously; and
R₅ is hydrogen, C₁-C₇-alkyl or unsubstituted or substituted phenyl;
A is —C(═O)—NH— with the —NH— bound to the ring comprising Q and Z in formula I or —NH—C(═O)— with the —C(═O)— bound to the ring comprising Q and Z in formula I;
Z is CH or N; and
Q is —S— or —CH═CH—;
or a salt thereof where one or more salt-forming groups are present.

2. A compound or formula I according to claim 1, wherein Q is —CH═CH— and R₁, R₂, R₃, R₄, R₅, A and Z are as defined in claim 1, or a—preferably pharmaceutically acceptable—salt thereof.

3. A compound of the formula I according to claim 1, wherein A is —C(═O)—NH—with the —NH— bound to the ring comprising Q and Z in formula I and R₁, R₂, R₃, R₄, R₅, Q and Z are as defined in claim 1, or a—preferably pharmaceutically acceptable—salt thereof.

4. A compound of the formula I according to claim 1, wherein one of R₁ and R₂ is hydrogen and the other is hydrogen or a moiety selected from the group consisting of 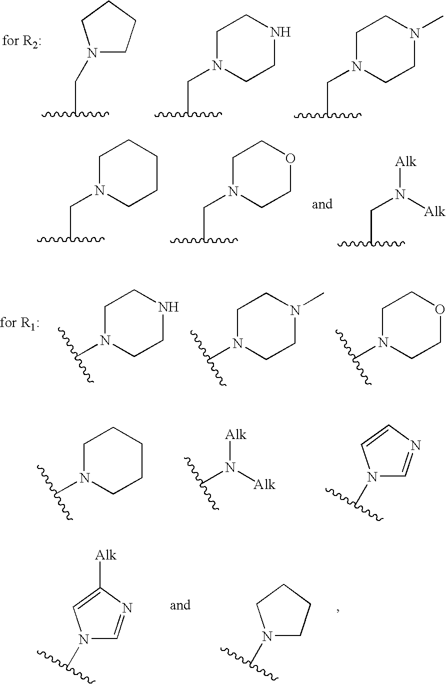
wherein “Alk” is alkyl, preferably lower alkyl, more preferably methyl or ethyl; and R₃, R₄, R₅, A, Q and Z are as defined in claim 1, or a—preferably pharmaceutically acceptable—salt thereof.

5. A compound of the formula I according to claim 1, wherein
each of R₁ and R₂ is hydrogen;
R₃ is C₁-C₇-alkyl, especially methyl;
R₄ is bicyclic heterocyclyl selected from the group consisting of 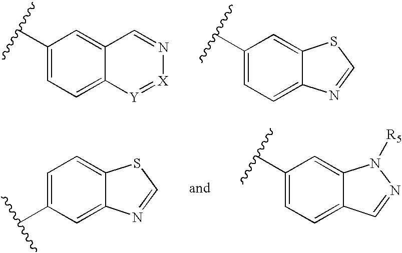
wherein
X is CH, N or C—NH₂;
Y is CH or N;
with the proviso that not both of X and Y are N simultaneously;
and R₅ is hydrogen, C₁-C₇-alkyl or phenyl;
(wherein R₄ is preferably 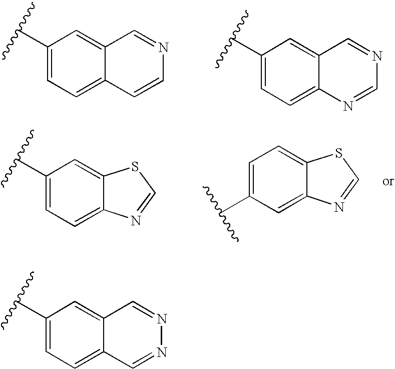
A is —C(═O)—NH— (with the —NH— bound to the ring comprising Q and Z in formula I) or —NH—C(═O)— (with the —C(═O)— bound to the ring comprising Q and Z in formula I);
Z is CH; and
Q is —CH═CH—;
or a—preferably pharmaceutically acceptable—salt thereof where one or more salt-forming groups are present.

6. A compound of the formula I according to claim 1, wherein R₄ is

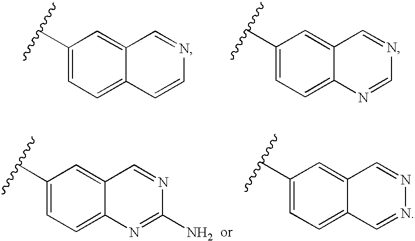

7. A compound of the formula I according to claim 1, selected from the group consisting of
N-(3-isoquinolin-7-yl-4-methyl-phenyl)-3-trifluoromethyl-benzamide,
N-(4-methyl-3-quinazolin-6-yl-phenyl)-3-trifluoromethyl-benzamide,
3-isoquinolin-7-yl-4-methyl-N-(3-trifluoromethyl-phenyl)-benzamide,
4-methyl-3-quinazolin-6-yl-N-(3-trifluoromethyl-phenyl)-benzamide,
N-(3-benzothiazol-6-yl-4-methyl-phenyl)-3-trifluoromethyl-benzamide,
3-benzothiazol-6-yl-4-methyl-N-(3-trifluoromethyl-phenyl)-benzamide,
N-(4-methyl-3-phthalazin-6-yl-phenyl)-3-trifluoromethyl-benzamide,
4-methyl-3-phthalazin-6-yl-N-(3-trifluoromethyl-phenyl)-benzamide,
N-(3-benzothiazol-5-yl-4-methyl-phenyl)-3-trifluoromethyl-benzamide,
3-benzothiazol-5-yl-4-methyl-N-(3-trifluoromethylphenyl)benzamide,
N-(3-Isoquinolin-7-yl-4-methyl-phenyl)-4-(4-methyl-piperazin-1-ylmethyl)-3-trifluoromethyl-benzamide,
3-Isoquinolin-7-yl-4-methyl-N-[4-(4-methyl-piperazin-1-ylmethyl)-3-trifluoromethyl-phenyl]-benzamide,
4-Methyl-N-[4-(4-methyl-piperazin-1-ylmethyl)-3-trifluoromethyl-phenyl]-3-(4,4,5,5-tetramethyl-[1,3,2]dioxaborolan-2-yl)-benzamide,
4-(4-Methyl-piperazin-1-ylmethyl)-N-(4-methyl-3-quinazolin-6-yl-phenyl)-3-trifluoromethyl-benzamide,
4-Methyl-N-[4-(4-methyl-piperazin-1-ylmethyl)-3-trifluoromethyl-phenyl]-3-quinazolin-6-yl-benzamide,
N-(4-Methyl-3-phthalazin-6-yl-phenyl)-4-(4-methyl-piperazin-1-ylmethyl)-3-trifluoromethyl-benzamide,
4-Methyl-N-[4-(4-methyl-piperazin-1-ylmethyl)-3-trifluoromethyl-phenyl]-3-phthalazin-6-yl-benzamide,
N-(4-Methyl-3-phthalazin-6-yl-phenyl)-4-piperidin-1-ylmethyl-3-trifluoromethyl-benzamide,
4-Dimethylaminomethyl-N-(3-isoquinolin-7-yl-4-methyl-phenyl)-3-trifluoromethyl-benzamide,
4-Dimethylaminomethyl-N-(4-methyl-3-phthalazin-6-yl-phenyl)-3-trifluoromethyl-benzamide,
N-(4-Methyl-3-phthalazin-6-yl-phenyl)-4-morpholin-4-ylmethyl-3-trifluoromethyl-benzamide,
N-(3-Isoquinolin-7-yl-4-methyl-phenyl)-4-morpholin-4-ylmethyl-3-trifluoromethyl-benzamide,
4-Methyl-3-phthalazin-6-yl-N-(4-piperidin-1-ylmethyl-3-trifluoromethyl-phenyl)-benzamide,
4-Methyl-N-(4-morpholin-4-ylmethyl-3-trifluoromethyl-phenyl)-3-phthalazin-6-yl-benzamide,
N-(4-Dimethylaminomethyl-3-trifluoromethyl-phenyl)-4-methyl-3-phthalazin-6-yl-benzamide,
4-Methyl-3-phthalazin-6-yl-N-(4-pyrrolidin-1-ylmethyl-3-trifluoromethyl-phenyl)-benzamide,
and N-(3-(2-Aminoquinazolin-6-yl)-4-methyl-phenyl)-4-(4-methyl-piperazin-1-ylmethyl)-3-trifluoromethyl-benzamide
or a pharmaceutically acceptable salt thereof where a salt-forming group is present.

8. A process for the manufacture of a compound of the formula I, or a salt thereof, according to claim 1, comprising reacting a boronic acid derivative of the formula II, 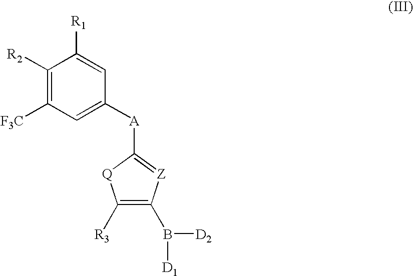
wherein D₁ and D₂ are hydroxy or substituted hydroxy, or together with the binding boron atom and two binding oxygen atoms form a ring of the formula IIA, 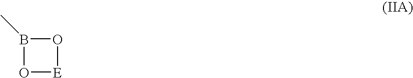
wherein E is alkylene, substituted alkylene, unsubstituted or substituted cycloalkylene, unsubstituted or substituted bicycloalkylene or unsubstituted or substituted tricycloalkylene,
with a coupling partner of the formula III,
R₄-L (III)
wherein R₄ is as defined according to claim 1 and L is a leaving group;
and, if desired, transforming a compound of formula I into a different compound of formula I, transforming a salt of an obtainable compound of formula I into the free compound or a different salt, and/or transforming an obtainable free compound of formula I into a salt thereof.

9. A pharmaceutical composition comprising a compound of the formula I, or a pharmaceutically acceptable salt thereof, according to claim 1 and a pharmaceutically acceptable carrier.

10. A compound of the formula I, or a pharmaceutically acceptable salt thereof, according to claim 1 for use in the diagnostic and/or therapeutic treatment of the animal, especially mammalian, or human body.

11. The use of a compound of the formula I, or a pharmaceutically acceptable salt thereof, according to claim 1 in the treatment, or for the preparation of a pharmaceutical preparation for the treatment of, one or more diseases or disorders that depend on one or more protein kinases, especially on one or more protein tyrosine kinases, especially selected from the group consisting of ephrin receptor kinases, preferably EphB4 kinase, c-abl, KDR, c-Src, c-raf, b-raf, Tie/Tek and KDR kinase; or a mutated variant thereof.

12. The use of a compound of the formula I, or a pharmaceutically acceptable salt thereof, according to claim 1 in the treatment, or for the manufacture of a pharmaceutical composition for the treatment, of a proliferative disease.

13. A method of treatment for a disease that responds to inhibition of a kinase and/or is a proliferative disease; which comprises administering a prophylactically or especially therapeutically effective amount of a compound of formula I, or a pharmaceutically acceptable salt thereof, according to claim 1, especially to a warm-blooded animal, for example a human, that, on account of one of the mentioned diseases, requires such treatment.

14. A combination comprising a therapeutically effective amount of a compound of the formula I, or a pharmaceutically acceptable salt thereof, according to claim 1 and a second drug substance, or a pharmaceutically acceptable salt thereof.
